(19) United States
(12) Patent Application Publication
(10) Pub. No.: US 2009/0281176 A1
(43) Pub. Date: Nov. 12, 2009

(54) PROCESS FOR THE SYNTHESIS OF RAMELTEON AND ITS INTERMEDIATES

(76) Inventors: Vinod Kumar Kansal, Haryana (IN); Dhirenkumar N. Mistry, Rajpipla-Gujarat (IN); Sanjay L. Vasoya, Gujarat (IN); Rakesh Patel, Gujarat (IN); Arpan M. Jadav, Gujarat (IN)

Correspondence Address: KENYON & KENYON LLP, ONE BROADWAY, NEW YORK, NY, 10004, US

(21) Appl. No.: 12/264,183

(22) Filed: Nov. 3, 2008

Related U.S. Application Data
(60) Provisional application No. 61/001,608, filed on Nov. 1, 2007.

(51) Int. Cl. A61K 31/343; C07D 307/80; C07D 413/02

(52) U.S. Cl. 514/468; 549/458; 548/230

(57) ABSTRACT

A process for the preparation of ramelteon and intermediates useful in the process. The process suitable for industrial scale provides increased yield and/or greater purity with fewer process steps.

This application claims priority of provisional application 61/001,608 filed Nov. 1, 2007, whose content is incorporated in its entirety herein by reference.

### FIELD OF THE INVENTION

The present invention relates to the synthesis of (S)—N-[2-(1,6,7,8-tetrahydro-2H-indeno-[5,4-b]furan-8-yl)ethyl] propionamide i.e. Ramelteon.

### BACKGROUND OF THE INVENTION

Ramelteon is a melatonin receptor agonist. The empirical formula for ramelteon is C₁₆H₂₁NO₂, and its molecular weight is 259.34. Ramelteon is freely soluble in methanol, ethanol DMSO (dimethylsulfoxide), 1-octanol and highly soluble in water and aqueous buffer. Ramelteon has the following chemical structure:

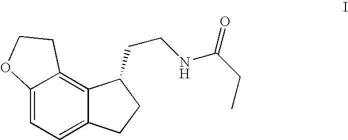

Ramelteon is the active ingredient in ROZEREM®, and is approved by the United States Food and Drug Administration for the treatment of insomnia characterized by difficulty with sleep onset.

Different processes for preparing (S)—N-[2-(1,6,7,8-tetrahydro-2H-indeno-[5,4-b]furan-8-yl)ethyl]propionamide i.e. Ramelteon are disclosed in U.S. Pat. No. 6,034,239, JP 11080106, JP 11140073 and WO 2006/030739.

U.S. Pat. No. 6,034,239 reports the following processes for the preparation of ramelteon:

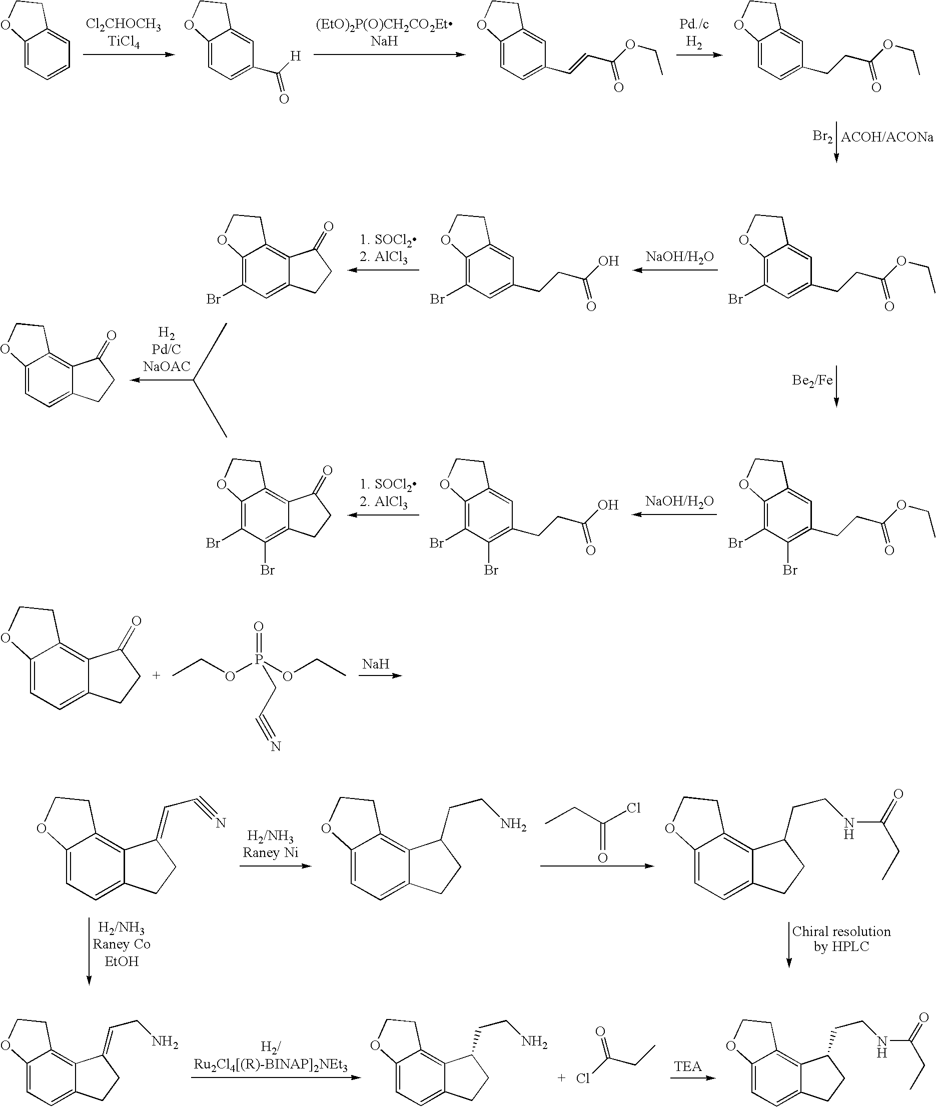

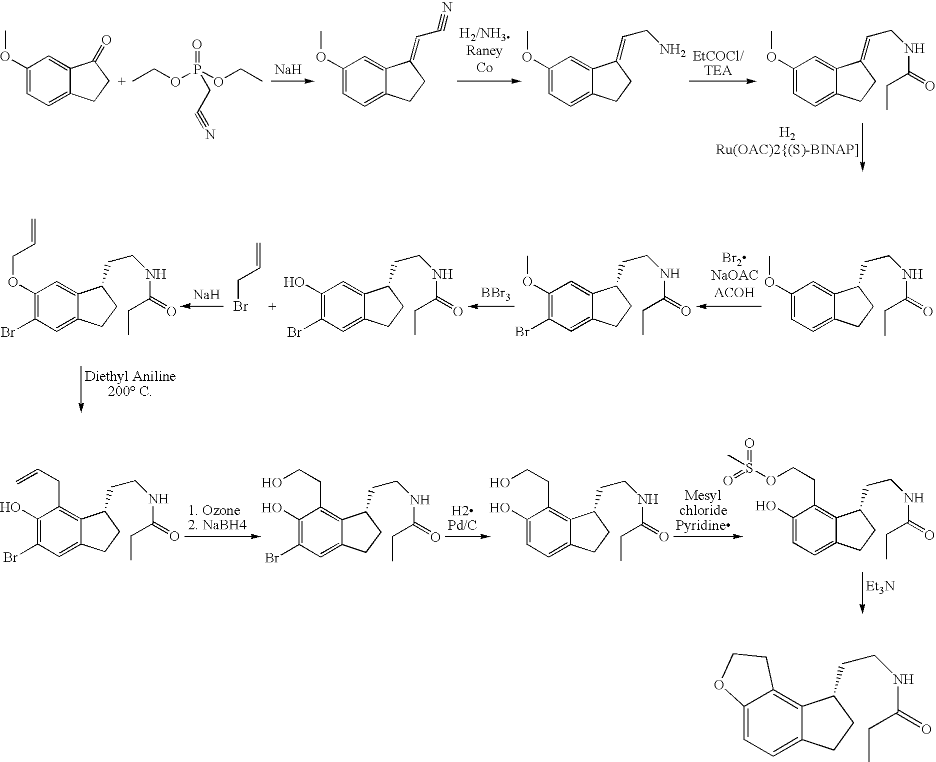

Japan Patent Publication no. 11080106 reports the following processes for the preparation of ramelteon:

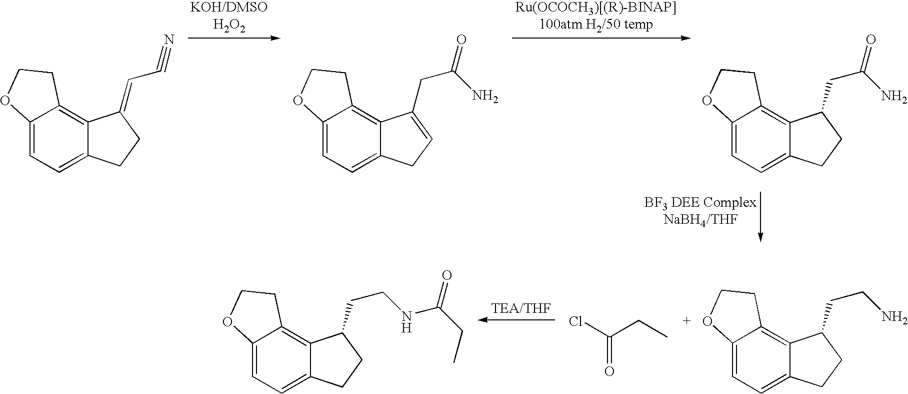

Japan Patent Publication no. 11140073 reports the following processes for the preparation of an intermediate of ramelteon:

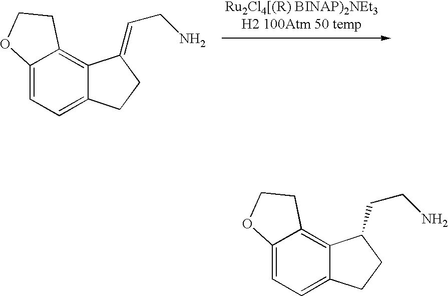

PCT Publication no. 2006/030739 reports the following processes for the preparation of ramelteon:

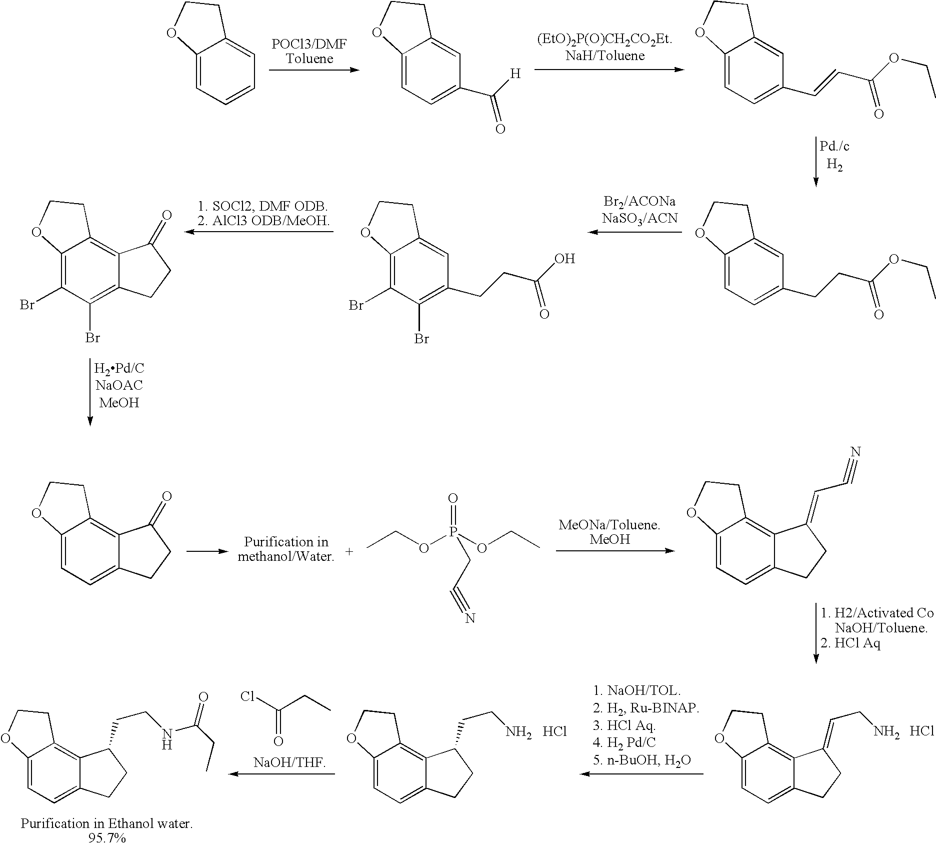

Provided is an alternate process, suitable for industrial scale for the preparation of ramelteon.

### SUMMARY OF INVENTION

In one embodiment, the present invention provides an intermediate, having the Formula III:

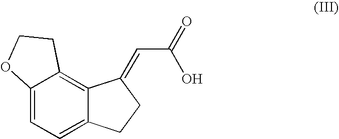

In another embodiment, the present invention encompasses a process for preparing the ramelteon intermediate of formula III, comprising hydrolyzing a compound of formula II.

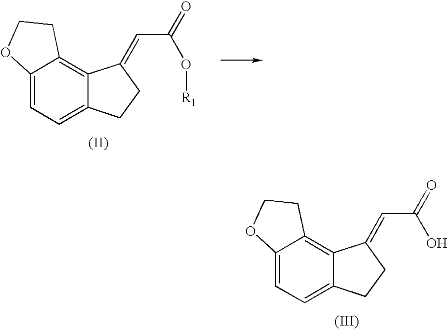

wherein R₁ is a C₁ to C₆ straight or branched alkyl, preferably C₁-C₄ straight or branched alkyl.

In one embodiment, the present invention encompasses a process for preparing ramelteon by preparing the compound of Formula III as described above, and converting it to ramelteon.

In another embodiment, the present invention provides a intermediate, having the Formula IV:

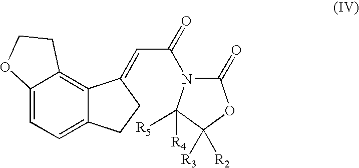

wherein R₂, R₃, R₄ and R₅ can be hydrogen, C₁-C₆ alkyl(preferably C₁-C₄ alkyl), C₆-C₁₂ aryl, or arylalkyl, wherein the alkyl group contains 1-4 carbon atoms and the aryl group contains 6-12 carbon atoms.

In another embodiment, the present invention encompasses a process for preparing the ramelteon intermediate of formula IV comprising condensation of III with a chiral compound:

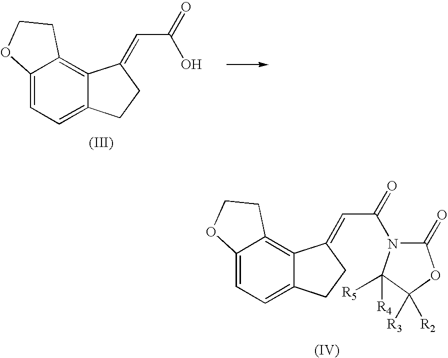

wherein R₂, R₃, R₄ and R₅ can be hydrogen, C₁-C₆ alkyl (preferably C₁-C₄ alkyl), C₆-C₁₂ aryl, or arylalkyl (wherein the alkyl group contains 1-4 carbon atoms and the aryl group contains 6-12 carbon atoms).

In one embodiment, the present invention encompasses a process for preparing ramelteon by preparing the compound of Formula IV as described above, and converting it to ramelteon.

In another embodiment, the present invention encompasses another process for preparing the ramelteon intermediate of formula IV, by condensation of the compound of V with a compound of formula VI:

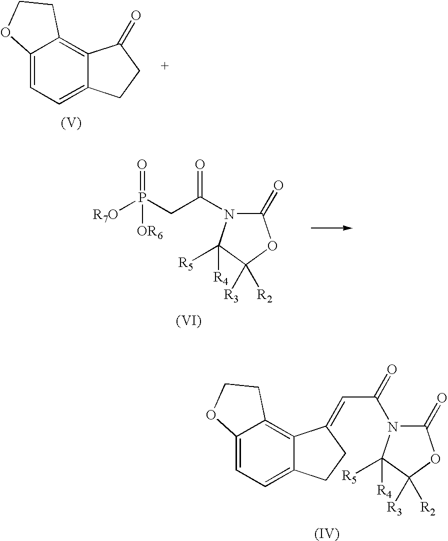

wherein R₂, R₃, R₄ and R₅ can be hydrogen, C₁-C₆ alkyl(preferably C₁-C₄ alkyl), C₆-C₁₂ aryl, or arylalkyl, wherein the alkyl group contains 1-4 carbon atoms and the aryl group contains 6-12 carbon atoms). R₆ and R₇ can be C₁-C₆ alkyl (preferably C₁-C₄ alkyl), or C₆-C₁₂ aryl.

In one embodiment, the present invention encompasses a process for preparing ramelteon by preparing the compound of Formula IV as described above, and converting it to ramelteon.

In another embodiment, the present invention provides an intermediate, having the Formula VII:

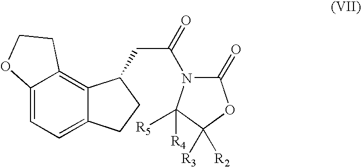

wherein R₂, R₃, R₄ and R₅ can be hydrogen, C₁-C₆ alkyl (preferably C₁-C₄ alkyl), C₆-C₁₂ aryl, or arylalkyl (wherein the alkyl group contains 1-4 carbon atoms and the aryl group contains 6-12 carbon atoms).

In another embodiment, the present invention encompasses a process for preparing the ramelteon intermediate of formula VII comprising reduction of a compound of IV in presence of catalyst.

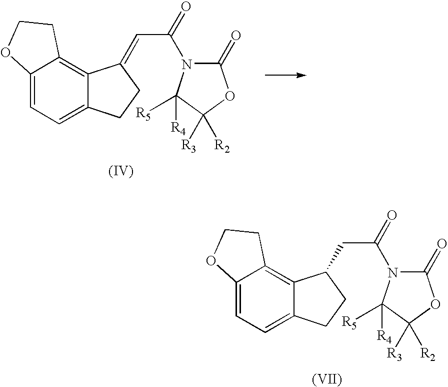

wherein R₂, R₃, R₄ and R₅ can be hydrogen, C₁-C₆ alkyl (preferably C₁-C₄ alkyl), C₆-C₁₂ aryl, or arylalkyl (wherein the alkyl group contains 1-4 carbon atoms and the aryl group contains 6-12 carbon atoms).

In one embodiment, the present invention encompasses a process for preparing ramelteon by preparing a compound of Formula VII as described above, and converting it to ramelteon.

In another embodiment, the present invention encompasses a process for preparing the ramelteon intermediate of Formula VIII:

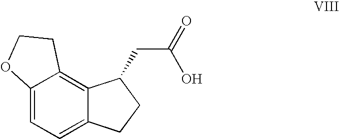

comprising hydrolyzing a compound of formula VII. The compound of formula VIII can be isolated as a free acid or as an amine addition salt,

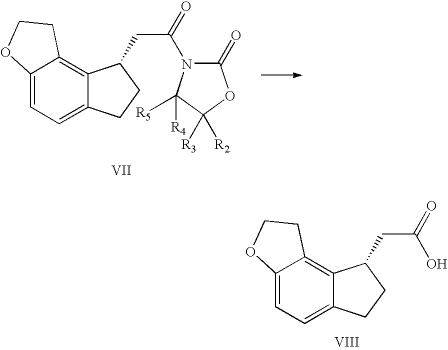

wherein R₂, R₃, R₄ and R₅ can be hydrogen, C₁-C₆ alkyl (preferably C₁-C₄ alkyl), C₆-C₁₂ aryl, or arylalkyl (wherein the alkyl group contains 1-4 carbon atoms and the aryl group contains 6-12 carbon atoms).

In one embodiment, the present invention encompasses a process for preparing ramelteon by preparing the compound of Formula VIII as described above, and converting it to ramelteon.

In one embodiment, the present invention encompasses a process for the preparation of ramelteon comprises the following steps:

(a) hydrolysis of the compound of formula II;

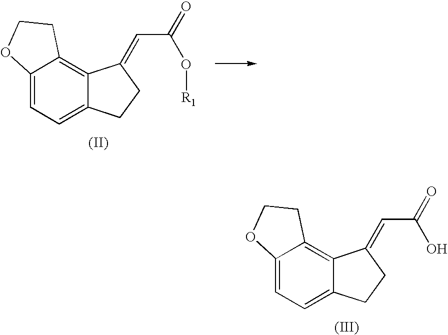

(b) condensation of the compound of formula III with a chiral compound to give a compound of formula IV:

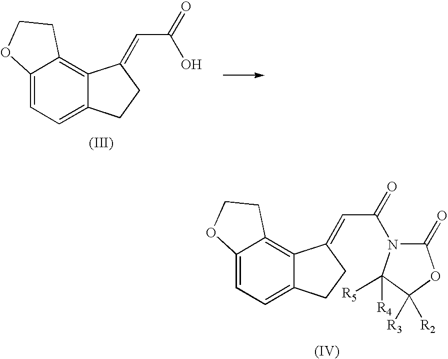

wherein R₂, R₃, R₄ and R₅ can be hydrogen, C₁-C₆ alkyl (preferably C₁-C₄ alkyl), C₆-C₁₂ aryl, or arylalkyl (wherein the alkyl group contains 1-4 carbon atoms and the aryl group contains 6-12 carbon atoms);
(c) hydrogenation of a compound of formula IV in presence of a catalyst to give a compound of VII:

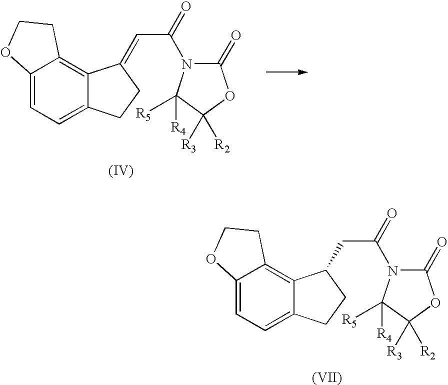

wherein R₂, R₃, R₄ and R₅ can be hydrogen, C₁-C₆ alkyl (preferably C₁-C₄ alkyl), C₆-C₁₂ aryl, or arylalkyl (wherein the alkyl group contains 1-4 carbon atoms and the aryl group contains 6-12 carbon atoms);
(d) hydrolysis of a compound of formula VII in the presence of base or acid:

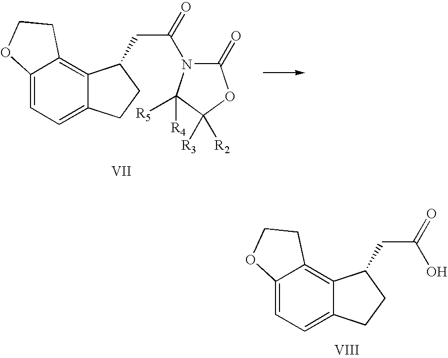

(e) amination of the compound of formula VIII to obtain the compound of formula IX:

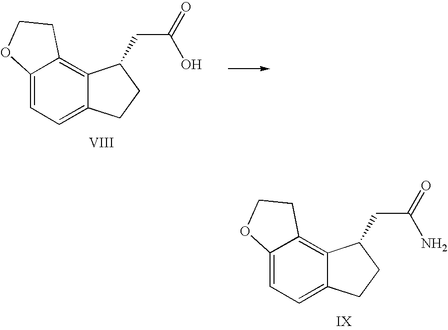

(f) reduction of the compound of formula IX with a ketone reducing agent to obtain the compound of formula X:

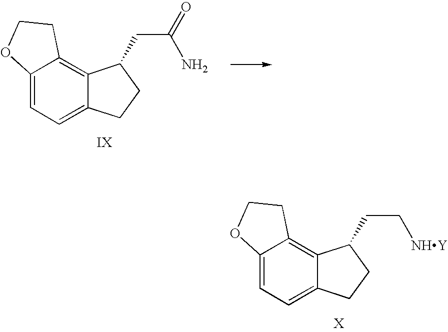

wherein Y is a pharmaceutically acceptable which includes oxalates, sulphates, nitrates, phosphates, perchlorates, borates, hydrohalides, acetates (such as trifluoroacetate), tartrates, maleates, citrates, fumarates, succinates, palmoates, methanesulphonates, benzoates, salicylates, benzenesulfonates, ascorbates, glycerophosphates, and ketoglutarates.
(g) condensation of a compound of formula X with propionyl chloride and a base to produce ramelteon of formula I,

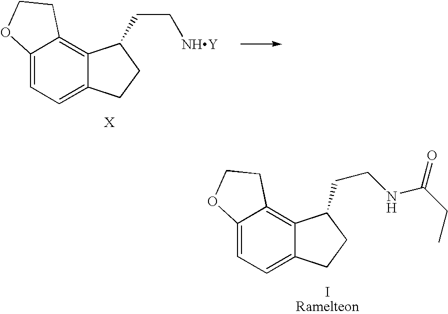

In one embodiment, the present invention encompasses a process for preparing ramelteon by preparing the compound of Formula VII as described above, and converting it to ramelteon.

In one embodiment the present invention provides a compound (Compound IV-a) the following structure:

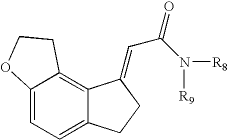

wherein R₈ and R₉ are substituted in such way to give an amine resulting from reaction with an amine or a cyanate selected from the group consisting of: (R)-1-Phenylethylamine, (S)-1-Phenylethylamine, (R)-1-(4-Methylphenyl)ethylamine, (S)-1-(4-Methylphenyl)ethylamine, (R)-1-(2-Methoxyphenyl)ethylamine, (S)-1-(2-Methoxyphenyl)ethylamine, (R)-1-(3-Methoxyphenyl)ethylamine, (S)-1-(3-Methoxyphenyl)ethylamine, (R)-1-(4-Methoxyphenyl)ethylamine, (S)-1-(4-Methoxyphenyl)ethylamine, (R)-1-(4-Chlorphenyl)ethylamine, (S)-1-(4-Chlorphenyl)ethylamine, (R)-1-(3-Chlorophenyl)ethylamine, (S)-1-(3-Chlorophenyl)ethylamine, (R)-1-(3-Bromophenyl)ethylamine, (S)-1-(3-Bromophenyl)ethylamine, (R)-1-(4-Bromophenyl)ethylamine, (S)-1-(4-Bromophenyl)ethylamine, (R)-1-(4-Fluorphenyl)ethylamine, (S)-1-(4-Fluorphenyl)ethylamine, (R)-1-(3,4-Dimethoxyphenyl)ethylamine, (S)-1-(3,4-Dimethoxyphenyl)ethylamine, (R)-1-(1-Naphthyl)ethylamine, (S)-1-(1-Naphthyl)ethylamine, (R)-1-Aminotetraline, (S)-1-Aminotetraline, (R)-1-Aminoindane,
(S)-1-Aminoindane, (R)-1-(2-Naphthyl)ethylamine, (S)-1-(2-Naphthyl)ethylamine, (R)-3-Methyl-2-butylamine, (S)-3-Methyl-2-butylamine, (R)-2-Hexylamine, (S)-2-Hexylamine, (R)-2-Heptylamine, (S)-2-Heptylamine, (R)-2-Octylamine, (S)-2-Octylamine, (R)-2-Nonylamine, (S)-2-Nonylamine, (R)-3,3-Dimethyl-2-aminobutane,
(S)-3,3-Dimethyl-2-aminobutane, (R)-1-Cyclopropylethylamine, (S)-1-Cyclopropylethylamine, (R)-1-Cyclohexylethylamine, (S)-1-Cyclohexylethylamine, (R)-1-Phenylpropylamine, (S)-1-Phenylpropylamine, (R)-1-Phenylbutylamine, (S)-1-Phenylbutylamine, (S)-1-Methoxy-2-aminopropane, (1R-trans)-2-(Phenylmethoxy)cyclopentaneamine, (1S-trans)-2-(Phenylmethoxy)cyclopentaneamine, (1R-trans)-2-(Phenylmethoxy)cyclohexaneamine, (1S-trans)-2-(Phenylmethoxy)cyclohexaneamine, (R)—N-Benzyl-1-phenylethylamine, (S)—N-Benzyl-1-phenylethylamine, (R,R)-Bis-(1-phenylethyl)amine, (S,S)-Bis-(1-phenylethyl)amine, (R)-1-Phenylethylisocyanate, (S)-1-Phenylethylisocyanate, (R)-1-Phenylethylhydroxylamine, and (S)-1-Phenylethylhydroxylamine.
In one embodiment, the present invention provides a process for the above compound comprising reacting compound of formula IV-a with an amine to get the compound of formula IV-a:

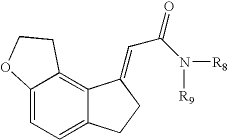

wherein each of R₈ and R₉ is independently selected from hydrogen, C₁-C₂₀ alkyl, C₆-C₁₂ aryl, alkylaryl (wherein the alkyl group contains 1-4 carbon atoms, and the aryl group contains 6-12 carbon atoms), optionally substituted with oxygen, nitrogen or halogen, wherein R₈ and R₉ are substituted in such a way so as to give a chiral compound. In one embodiment, each of R₈ or R₉ is substituted in such way to provide an amine selected from the group consisting of: (R)-1-Phenylethylamine, (S)-1-Phenylethylamine, (R)-1-(4-Methylphenyl)ethylamine, (S)-1-(4-Methylphenyl)ethylamine, (R)-1-(2-Methoxyphenyl)ethylamine, (S)-1-(2-Methoxyphenyl)ethylamine, (R)-1-(3-Methoxyphenyl)ethylamine, (S)-1-(3-Methoxyphenyl)ethylamine, (R)-1-(4-Methoxyphenyl)ethylamine, (S)-1-(4-Methoxyphenyl)ethylamine, (R)-1-(4-Chlorphenyl)ethylamine, (S)-1-(4-Chlorphenyl)ethylamine, (R)-1-(3-Chlorophenyl)ethylamine, (S)-1-(3-Chlorophenyl)ethylamine, (R)-1-(3-Bromophenyl)ethylamine, (S)-1-(3-Bromophenyl)ethylamine, (R)-1-(4-Bromophenyl)ethylamine, (S)-1-(4-Bromophenyl)ethylamine, (R)-1-(4-Fluorphenyl)ethylamine, (S)-1-(4-Fluorphenyl)ethylamine, (R)-1-(3,4-Dimethoxyphenyl)ethylamine, (S)-1-(3,4-Dimethoxyphenyl)ethylamine, (R)-1-(1-Naphthyl)ethylamine, (S)-1-(1-Naphthyl)ethylamine, (R)-1-Aminotetraline, (S)-1-Aminotetraline, (R)-1-Aminoindane, (S)-1-Aminoindane, (R)-1-(2-Naphthyl)ethylamine, (S)-1-(2-Naphthyl)ethylamine, (R)-3-Methyl-2-butylamine, (S)-3-Methyl-2-butylamine, (R)-2-Hexylamine, (S)-2-Hexylamine, (R)-2-Heptylamine, (S)-2-Heptylamine, (R)-2-Octylamine, (S)-2-Octylamine, (R)-2-Nonylamine, (S)-2-Nonylamine, (R)-3,3-Dimethyl-2-aminobutane, (S)-3,3-Dimethyl-2-aminobutane, R)-1-Cyclopropylethylamine, (S)-1-Cyclopropylethylamine, (R)-1-Cyclohexylethylamine, (S)-1-Cyclohexylethylamine, (R)-1-Phenylpropylamine, (S)-1-Phenylpropylamine, (R)-1-Phenylbutylamine, (S)-1-Phenylbutylamine, (S)-1-Methoxy-2-aminopropane, (1R-trans)-2-(Phenylmethoxy)cyclopentaneamine, (1S-trans)-2-(Phenylmethoxy)cyclopentaneamine, (1R-trans)-2-(Phenylmethoxy)cyclohexaneamine, (1S-trans)-2-(Phenylmethoxy)cyclohexaneamine, (R)—N-Benzyl-1-phenylethylamine, (S)—N-Benzyl-1-phenylethylamine, (R)-1-Phenylethylisocyanate, (S)-1-Phenylethylisocyanate, (R,R)-Bis-(1-phenylethyl)amine, (S,S)-Bis-(1-phenylethyl)amine, and (R)-1-Phenylethylhydroxylamine, (S)-1-Phenylethylhydroxylamine.

In one embodiment, the present invention provides a compound having the following formula:

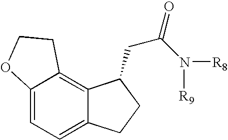

wherein R₈ and R₉ are substituted in such way to give an amine resulting from reaction with an amine or a cyanate selected from the group consisting of: (R)-1-Phenylethylamine, (S)-1-Phenylethylamine, (R)-1-(4-Methylphenyl)ethylamine, (S)-1-(4-Methylphenyl)ethylamine, (R)-1-(2-Methoxyphenyl)ethylamine, (S)-1-(2-Methoxyphenyl)ethylamine, (R)-1-(3-Methoxyphenyl)ethylamine, (S)-1-(3-Methoxyphenyl)ethylamine, (R)-1-(4-Methoxyphenyl)ethylamine, (S)-1-(4-Methoxyphenyl)ethylamine, (R)-1-(4-Chlorphenyl)ethylamine, (S)-1-(4-Chlorphenyl)ethylamine, (R)-1-(3-Chlorophenyl)ethylamine, (S)-1-(3-Chlorophenyl)ethylamine, (R)-1-(3-Bromophenyl)ethylamine, (S)-1-(3-Bromophenyl)ethylamine, (R)-1-(4-Bromophenyl)ethylamine, (S)-1-(4-Bromophenyl)ethylamine, (R)-1-(4-Fluorphenyl)ethylamine, (S)-1-(4-Fluorphenyl)ethylamine, (R)-1-(3,4-Dimethoxyphenyl)ethylamine, (S)-1-(3,4-Dimethoxyphenyl)ethylamine, (R)-1-(1-Naphthyl)ethylamine, (S)-1-(1-Naphthyl)ethylamine, (R)-1-Aminotetraline, (S)-1-Aminotetraline, (R)-1-Aminoindane, (S)-1-Aminoindane, (R)-1-(2-Naphthyl)ethylamine, (S)-1-(2-Naphthyl)ethylamine, R)-3-Methyl-2-butylamine, (S)-3-Methyl-2-butylamine, (R)-2-Hexylamine, (S)-2-Hexylamine, (R)-2-Heptylamine, (S)-2-Heptylamine, (R)-2-Octylamine, (S)-2-Octylamine, (R)-2-Nonylamine, (S)-2-Nonylamine, (R)-3,3-Dimethyl-2-aminobutane, (S)-3,3-Dimethyl-2-aminobutane, R)-1-Cyclopropylethylamine, (S)-1-Cyclopropylethylamine, (R)-1-Cyclohexylethylamine, (S)-1-Cyclohexylethylamine, (R)-1-Phenylpropylamine, (S)-1-Phenylpropylamine, (R)-1-Phenylbutylamine, (S)-1-Phenylbutylamine, (S)-1-Methoxy-2-aminopropane, (1R-trans)-2-(Phenylmethoxy)cyclopentaneamine, (1S-trans)-2-(Phenylmethoxy)cyclopentaneamine, (1R-trans)-2-(Phenylmethoxy)cyclohexaneamine, (1S-trans)-2-(Phenylmethoxy)cyclohexaneamine, (R)—N-Benzyl-1-phenylethylamine, (S)—N-Benzyl-1-phenylethylamine, (R)-1-Phenylethylisocyanate, (S)-1-Phenylethylisocyanate, (R,R)-Bis-(1-phenylethyl)amine, (S,S)-Bis-(1-phenylethyl)amine, and (R)-1-Phenylethylhydroxylamine, (S)-1-Phenylethylhydroxylamine.

In one embodiment, the present invention provides a process for preparing the compound of formula:

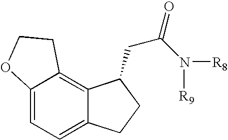

comprising reducing the alkene bond of a compound of formula IV:

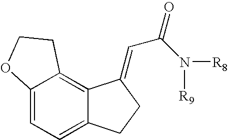

wherein each of R₈ and R₉ is independently selected from hydrogen, C₁-C₂₀ alkyl, C₆-C₁₂ aryl, alkylaryl (wherein the alkyl group contains 1-4 carbon atoms, and the aryl group contains 6-12 carbon atoms), optionally substituted with oxygen, nitrogen or halogen, wherein R₈ and R₉ are substituted in such a way so as to give a chiral compound.

### DETAILED DESCRIPTION OF THE INVENTION

As used herein, the term “alkoxy” denotes alkyl group as defined above attached via an oxygen linkage to the rest of the molecule. Representative examples of those groups are —OCH₃, —OC₂H₅ and the like. The alkyl group is typically a C₁-C₆ preferably C₁-C₄ group.

As used herein, ammonia is aqueous ammonia, liquid ammonia and gaseous ammonia. Aqueous ammonia is preferably about 5-35% aqueous ammonia by weight.

As used herein, the term “halogenated hydrocarbons” refers to cyclic or acyclic, saturated or unsaturated aliphatic or aromatic hydrocarbons that are substituted with one or more (preferably from 1-6) halogen atoms (preferably fluoro or chloro or a mixture of both). Examples of halogenated hydrocarbons include, but are not limited to, halogenated alkanes such as chloromethane, dichloromethane, chloroethane, dichlorotrifluoroethane, difluoroethane, hexachloroethane, pentafluoroethane, halogenated alkenes such as such as tetrachloroethene, dichloroethene, trichloroethene, vinyl chloride, chloro-1,3-butadiene, chlorotrifluoroethylene, or halogenated benzenes such as benzotrichloride, benzyl chloride, bromobenzene, chlorobenzene, chlorotoluene, dichlorobenzene, fluorobenzene, or trichlorobenzene. The preferred halogen is chlorine. The preferred halogenated hydrocarbons are aromatic hydrocarbons (preferably C₆ to C₁₂) or C₁-C₆ alkanes, and more preferably chlorinated aromatic hydrocarbons (preferably C₆ to C₁₂) or C₁-C₄ alkanes. The more preferred halogenated hydrocarbons are chlorobenzene, o- or p-dichlorobenzene, dichloromethane, or o-chlorotoluene.

All the reactions that are described below are maintained for a sufficient period of time to obtain the desired product. One of ordinary skilled in the art could easily monitor the reactions to determine whether a sufficient period of time has elapsed using TLC techniques.

In one embodiment, the present invention provides a compound of formula III, which is a ramelteon intermediate:

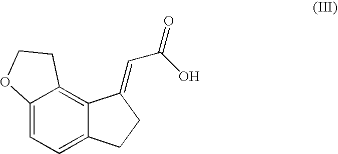

This intermediate can be characterized by the following NMR data:

¹H NMR: 7.0457-7.0660 (Ar—H, 1H, dd), 6.7716-6.7918 (Ar—H, 1H, dd), 6.0550-6.0676 (CH, 1H,t), 4.5831-4.6270 (CH2, 2H, m), 3.2895-3.3688 (CH2, 2H, t), 3.2520-3.2895 (CH2, 2H, m,) 2.9468-2.9771 (CH2, 2H, t)

¹³C NMR: 29.81, 30.15, 32.17, 59.94, 110.62, 112.13, 122.07, 124.60, 137.04, 142.20, 160.03, 163.72, 167.66

Compound III can be present in isolated or purified form, such as by HPLC, from other compounds (including Compound II and/or ramelteon). Compound III can be in a mixture (composition) comprising at least about 20% or at least about 40%, or at least about 60% or at least about 80% by weight of compound III, and preferably at least about 90%, more preferably at least about 95% by weight of compound III.

In another embodiment, the present invention encompasses a process for preparing the compound III, comprising hydrolyzing a compound of formula II:

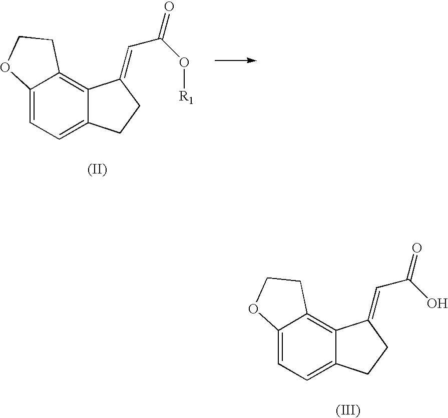

wherein R₁ is C₁ to C₄ straight or branched alkyl; preferably R₁ is methyl or ethyl.

Compound II can be prepared, for example, according to the procedure described in PCT application no. PCT/US08/65590.

This process is preferably conducted in the presence of an acid or a base for hydrolysis is selected from alkali metal carbonates, hydroxides or hydrides, for example potassium bicarbonate, sodium bicarbonate, potassium carbonate, sodium carbonate, sodium hydroxide, potassium hydroxide, sodium hydride and potassium hydride; most preferably sodium hydroxide. Preferably, the acid is selected from sulfuric acid, hydrochloric acid.

The hydrolysis can be carried out by combining compound II, a water miscible solvent, preferably a C₁-C₅ alcohol such as methanol, water, and a base or an acid as specified above. The reaction mixture is then maintained until hydrolysis is completed, such as for about 2 to about 10 hours, preferably for about 4 hours to about 6 hours. An additional amount of water can then be added to aid in recovery of the product. This aqueous phase can be washed with water immiscible solvent to remove impurities. The product can be recovered from the aqueous phase by precipitation, which can be achieved by adding an acid if the hydrolysis was carried out in a base, or vice versa. The product can be isolated by typical techniques such as filtration, and be optionally dried, such as at a temperature of about 40° C. to about 80° C.

Compound of Formula III as described above can be converted to one of the intermediates below and to ramelteon.

In one embodiment, the present invention provides a compound of formula IV, which is a ramelteon:

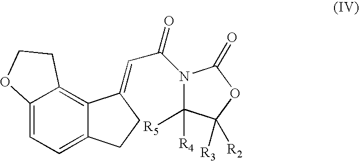

wherein R₂, R₃, R₄ and R₅ can be hydrogen, C₁-C₆ alkyl (preferably C₁-C₄ alkyl), C₆-C₁₂ aryl, or arylalkyl (wherein the alkyl group contains 1-4 carbon atoms, and the aryl group contains 6-12 carbon atoms). Each of R₂, R₃, R₄ and R₅ are selected in such way as to obtain a chiral compound, preferably R₂, R₃, R₄ and R₅ are substituted in such way so that 4- or 5-phenyl-2-oxazolidinone is formed.

Compound IV can be present in isolated or purified form, such as by HPLC, from other compounds (including Compound III, the amine used in the reaction and/or ramelteon). Compound IV can be in a mixture (composition) comprising at least about 20% or at least about 40%, or at least about 60% or at least about 80% by weight of compound IV.

This compound (when 4-phenyl-2-oxazolidinone is used: R₂, R₃, and R₄=hydrogen, R₅=phenyl) can be characterized by the following NMR data:

¹H NMR: (CDCl₃, 300 MHz): 6.855-6.956 (Ar—H, 1H, dd), 7.102-7.122 (Ar—H, 1H, dd), 7.334-7.438 (Ar-Ph, 5H, m), 7.728-7.740 (—CH, 1H, t), 5.572-5.603 (—CH, 1H, q), 4.6744.792 (—CH2, 2H, m), 4.296-4.306 (—CH2, 2H, m), 3.549-3.571 (—CH2, 2H, m), 3.011-3.295 (—CH2, 2H, m), 2.981-3.011 (—CH2, 2H, m)

¹³C NMR: (CDCl₃, 75 MHz): 29.69, 30.10, 32.96, 57.74, 69.77, 71.70, 93.64, 109.02, 112.62, 122.58, 124.40, 125.84, 128.56, 129.22, 137.34, 139.60, 142.61, 154.13, 160.03, 164.61, 167.13

Ramelteon intermediate of formula IV, can be prepared by condensation of the compound of formula III with a chiral compound:

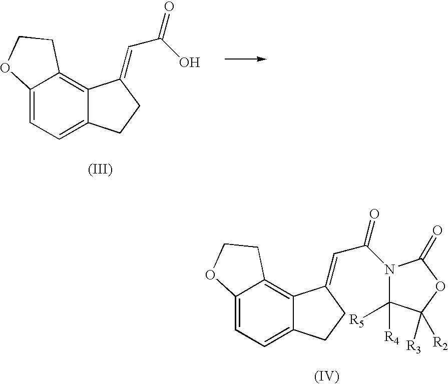

wherein R₂, R₃, R₄ and R₅ can be hydrogen, C₁-C₆ alkyl (preferably C₁-C₄ alkyl), C₆-C₁₂ aryl, or arylalkyl (wherein the alkyl group contains 1-4 carbon atoms, and the aryl group contains 6-12 carbon atoms

This reaction is preferably carried out in the presence of an organic base. The organic base is preferably an organic amine that has the formula N(Rₓ)(R_{y})(R_{z}), wherein (Rₓ), (R_{y}) and (R_{z}) are each independently selected from C₁-C₈ alkyl group, more preferably C₁-C₄ alkyl group, C₆-C₁₂ aryl group, or alkylaryl (wherein the alkyl group contains 1-4 carbon atoms, and the aryl group contains 6-12 carbon atoms). Examples of such bases include triethylamine, trimethylamine, diisoproyl ethyl amine, tributyl amine, tripropylamine, more preferably, triethyl amine. Acid chloride can be added to the reaction mixture. The acid chloride can be pivaloyl chloride, methane sulfonyl chloride, p-toluene sulfonyl chloride, n-butyl chloride. More preferably pivaloyl chloride can be used. Acid chloride can be added at a lower temperature, ranging from about −5° C. to about −25° C., preferably from about 0° C. to about 5° C. Then the chiral compound can be added. The chiral compound can be variously substituted 4-phenyl-2-oxazolidinone, preferably, the chiral compound is 4-phenyl-2-oxazolidinone. A catalytic amount of 4-dimethyl amino pyridine, diethylamino pyridine, and/or dipropyl amino pyridine is added. A catalytic amount is less than one mole and can be an amount of about 0.001 mol to about 0.2 mol. Preferably, 4-dimethyl amino pyridine is added. The reaction proceeds for about 30-40 minutes. The temperature of the reaction can be from about 10° C. to about 35° C., more preferably about 20° C. to about 25° C.

Other chiral compounds, particularly amines and cyanates, can be reacted to obtain an amine similar to compound IV. Examples of such amines and cyanates are:

(R)-1-Phenylethylamine, (S)-1-Phenylethylamine, (R)-1-(4-Methylphenyl)ethylamine, (S)-1-(4-Methylphenyl)ethylamine, (R)-1-(2-Methoxyphenyl)ethylamine, (S)-1-(2-Methoxyphenyl)ethylamine, (R)-1-(3-Methoxyphenyl)ethylamine, (S)-1-(3-Methoxyphenyl)ethylamine, (R)-1-(4-Methoxyphenyl)ethylamine, (S)-1-(4-Methoxyphenyl)ethylamine, (R)-1-(4-Chlorphenyl)ethylamine, (S)-1-(4-Chlorphenyl)ethylamine, (R)-1-(3-Chlorophenyl)ethylamine, (S)-1-(3-Chlorophenyl)ethylamine, (R)-1-(3-Bromophenyl)ethylamine, (S)-1-(3-Bromophenyl)ethylamine, (R)-1-(4-Bromophenyl)ethylamine, (S)-1-(4-Bromophenyl)ethylamine, (R)-1-(4-Fluorphenyl)ethylamine, (S)-1-(4-Fluorphenyl)ethylamine, (R)-1-(3,4-Dimethoxyphenyl)ethylamine, (S)-1-(3,4-Dimethoxyphenyl)ethylamine, (R)-1-(1-Naphthyl)ethylamine, (S)-1-(1-Naphthyl)ethylamine, (R)-1-Aminotetraline, (S)-1-Aminotetraline, (R)-1-Aminoindane,
(S)-1-Aminoindane, (R)-1-(2-Naphthyl)ethylamine, (S)-1-(2-Naphthyl)ethylamine, (R)-3-Methyl-2-butylamine, (S)-3-Methyl-2-butylamine, (R)-2-Hexylamine, (S)-2-Hexylamine, (R)-2-Heptylamine, (S)-2-Heptylamine, (R)-2-Octylamine, (S)-2-Octylamine, (R)-2-Nonylamine, (S)-2-Nonylamine, (R)-3,3-Dimethyl-2-aminobutane,
   (S)-3,3-Dimethyl-2-aminobutane, R)-1-Cyclopropylethylamine, (S)-1-Cyclopropylethylamine, (R)-1-Cyclohexylethylamine, (S)-1-Cyclohexylethylamine, (R)-1-Phenylpropylamine, (S)-1-Phenylpropylamine, (R)-1-Phenylbutylamine, (S)-1-Phenylbutylamine, (S)-1-Methoxy-2-aminopropane, (1R-trans)-2-(Phenylmethoxy)cyclopentaneamine, (1S-trans)-2-(Phenylmethoxy)cyclopentaneamine, (1R-trans)-2-(Phenylmethoxy)cyclohexaneamine, (1S-trans)-2-(Phenylmethoxy)cyclohexaneamine, (R)—N-Benzyl-1-phenylethylamine, (S)—N-Benzyl-1-phenylethylamine, (R)-1-Phenylethylisocyanate, (S)-1-Phenylethylisocyanate, (R,R)-Bis-(1-phenylethyl)amine, (S,S)-Bis-(1-phenylethyl)amine, and (R)-1-Phenylethylhydroxylamine, (S)-1-Phenylethylhydroxylamine.

Compound IV-a obtained by reaction with of compound III with amines and cyanates listed above has the following structure:

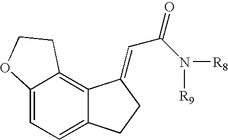

wherein each of R₈ and R₉ is independently selected from hydrogen, C₁-C₂₀ alkyl, aryl, alkylaryl, (wherein the alkyl group contains 1-4 carbon atoms, and the aryl group contains 6-12 carbon atoms), optionally substituted with oxygen, nitrogen or halogen, wherein R₈ and R₉ are substituted in such a way so as to give a chiral compound.

Compound IV-a can be present in isolated or purified form, such as by HPLC, from other compounds (including Compound III, the amine used in the reaction and/or ramelteon). Compound IV-a can be in a mixture (composition) comprising at least about 20% or at least about 40%, or at least about 60% or at least about 80% by weight of compound IV-a.

The compound of formula IV may also be prepared by a Wittig-Horner reaction, in particular, the reaction can be carried out by condensation of the compound of formula V with a compound of formula VI, preferably in the presence of a base.

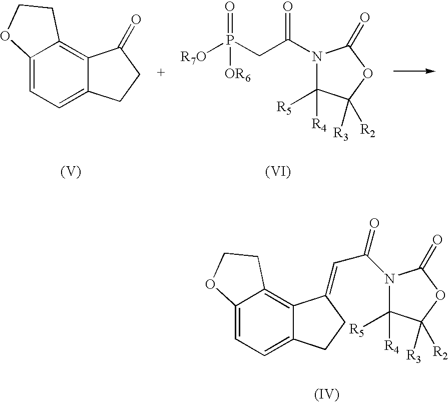

R₆ and R₇ are selected so that the optimal Wittig-Horner reaction would occur. Preferably R₆ and R₇ are C₁-C₃ alkyl group, or C₆-C₈ aryl group more preferably ethyl.

Compound V can be prepared, for example, according to the procedure described in PCT application no. PCT/US08/65590. Compound VI can be prepared, for example, according to the procedure described in J. Am. Chem. Soc. Vol. 120, No. 47, 1998, 12237-12254.

The reaction is preferably carried out in the presence of a solvent. The solvent can be an aromatic hydrocarbon (such as C₆-C₁₂), a cyclic ether (such as C₄-C₁₀), a polar aprotic solvent (such as C₁-C₈), ketones (such as C₃-C₈) and mixtures thereof. Aromatic hydrocarbons include toluene and xylene. Toluene is preferred. Cyclic ethers include dioxane, tetrahydrofuran, and tetrahydropyran. Tetrahydrofuran is preferred. DMF (dimethylformamide) and/or DMSO (dimethylsulfoxide) can be used as polar aprotic solvents. The reaction is carried out in the presence of a base. The base can be alkali metal hydride, or alkali hydroxide. Alkali metal hydrides include sodium hydride, potassium hydride, and sodium hydride is preferred. Alkali metal hydroxides can be sodium hydroxide, potassium hydroxide, and lithium hydroxide.

The above process can also comprise condensation of a compound of formula V

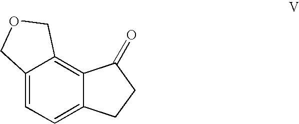

with a compound of formula VI-a

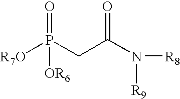

wherein each of R₈ and R₉ is independently selected from hydrogen, C₁-C₂₀ alkyl, C₆-C₁₂ aryl, alkylaryl (wherein the alkyl group contains 1-4 carbon atoms, and the aryl group contains 6-12 carbon atoms), optionally substituted with oxygen, nitrogen or halogen, wherein R₈ and R₉ are substituted in such a way so as to give a chiral compound. In one embodiment, R₈ and R₉ are substituted in such way to give an amine resulting from reaction with an amine or a cyanate selected from the group consisting of (R)-1-Phenylethylamine, (S)-1-Phenylethylamine, (R)-1-(4-Methylphenyl)ethylamine, (S)-1-(4-Methylphenyl)ethylamine, (R)-1-(2-Methoxyphenyl)ethylamine, (S)-1-(2-Methoxyphenyl)ethylamine, (R)-1-(3-Methoxyphenyl)ethylamine, (S)-1-(3-Methoxyphenyl)ethylamine, (R)-1-(4-Methoxyphenyl)ethylamine, (S)-1-(4-Methoxyphenyl)ethylamine, (R)-1-(4-Chlorphenyl)ethylamine, (S)-1-(4-Chlorphenyl)ethylamine, (R)-1-(3-Chlorophenyl)ethylamine, (S)-1-(3-Chlorophenyl)ethylamine, (R)-1-(3-Bromophenyl)ethylamine, (S)-1-(3-Bromophenyl)ethylamine, (R)-1-(4-Bromophenyl)ethylamine, (S)-1-(4-Bromophenyl)ethylamine, (R)-1-(4-Fluorphenyl)ethylamine, (S)-1-(4-Fluorphenyl)ethylamine, (R)-1-(3,4-Dimethoxyphenyl)ethylamine, (S)-1-(3,4-Dimethoxyphenyl)ethylamine, (R)-1-(1-Naphthyl)ethylamine, (S)-1-(1-Naphthyl)ethylamine, (R)-1-Aminotetraline, (S)-1-Aminotetraline, (R)-1-Aminoindane, (S)-1-Aminoindane, (R)-1-(2-Naphthyl)ethylamine, (S)-1-(2-Naphthyl)ethylamine, (R)-3-Methyl-2-butylamine, (S)-3-Methyl-2-butylamine, (R)-2-Hexylamine, (S)-2-Hexylamine, (R)-2-Heptylamine, (S)-2-Heptylamine, (R)-2-Octylamine, (S)-2-Octylamine, (R)-2-Nonylamine, (S)-2-Nonylamine, (R)-3,3-Dimethyl-2-aminobutane, (S)-3,3-Dimethyl-2-aminobutane, (R)-1-Cyclopropylethylamine, (S)-1-Cyclopropylethylamine, (R)-1-Cyclohexylethylamine, (S)-1-Cyclohexylethylamine, (R)-1-Phenylpropylamine, (S)-1-Phenylpropylamine, (R)-1-Phenylbutylamine, (S)-1-Phenylbutylamine, (S)-1-Methoxy-2-aminopropane, (1R-trans)-2-(Phenylmethoxy)cyclopentaneamine, (1S-trans)-2-(Phenylmethoxy)cyclopentaneamine, (1R-trans)-2-(Phenylmethoxy)cyclohexaneamine, (1S-trans)-2-(Phenylmethoxy)cyclohexaneamine, (R)—N-Benzyl-1-phenylethylamine, (S)—N-Benzyl-1-phenylethylamine, (R)-1-Phenylethylisocyanate, (S)-1-Phenylethylisocyanate, (R,R)-Bis-(1-phenylethyl)amine, (S,S)-Bis-(1-phenylethyl)amine, and (R)-1-Phenylethylhydroxylamine, (S)-1-Phenylethylhydroxylamine.

Compounds of Formula II, III, IV, IV-a, VI, VI-a, VII, and VIII as described above can be converted to one of the intermediates below and to ramelteon.

In one embodiment, the present invention provides a compound of formula VII, which is a ramelteon intermediate:

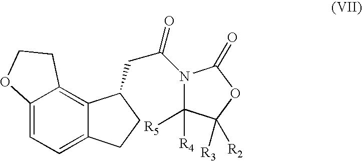

wherein R₂, R₃, R₄ and R₅ can be hydrogen, C₁-C₆ alkyl (preferably C₁-C₄ alkyl), C₆-C₁₂ aryl, or arylalkyl (wherein the alkyl group contains 1-4 carbon atoms, and the aryl group contains 6-12 carbon atoms). Each of R₂, R₃, R₄ and R₅ are selected in such way as to obtain a chiral compound, preferably R₂, R₃, R₄ and R₅ are substituted in such a was so that a 4- or 5-phenyl-2-oxazolidinone is formed.

Compound VII can be present in isolated or purified form, such as by HPLC, from other compounds (including Compound IV, the amine used in the reaction and/or ramelteon). Compound VII can be in a mixture (composition) comprising at least about 20% or at least about 40%, or at least about 60% or at least about 80% by weight of compound VII, and preferably at least about 90%, more preferably at least about 95% by weight of compound VII.

This compound (by reaction with 4-phenyl-2-oxazolidinone: R₂, R₃, and R₄=hydrogen, R₅=phenyl)) can be characterized by the following NMR data:

¹H NMR: (CDCl₃, 300 MHz): 6.618-6.638 (Ar—H, 1H, dd), 6.940-6.960 (Ar—H, 1H, dd), 7.359-7.450 (Ar-Ph, 5H, m), 5.477-5.508 (—CH, 1H, q), 4.6744.792 (—CH2, 2H, m), 4.296-4.306 (—CH2, 2H, m), 3.549-3.571 (CH2, 2H, m), −3.011-3.295 (CH2, 2H, m), 2.981-3.011 (—CH2, 2H, m),

¹³C NMR: (CDCl₃, 75 MHz): 28.41 30.30, 32.00, 39.58, 40.06, 57.62, 70.01, 71.26, 107.65, 117.32, 122.44, 123.35, 126.20, 128.87, 129.21, 135.82, 139.09, 142.04, 153.70, 156.96, 159.42, 171.69

In another embodiment, the present invention encompasses a process for preparing the ramelteon intermediate of formula VII by reduction of the double bond.

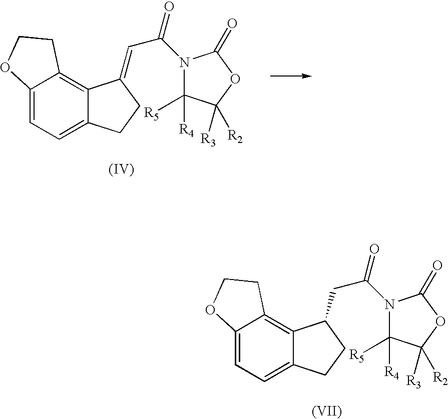

wherein R₂, R₃, R₄ and R₅ can be hydrogen, C₁-C₆ alkyl (preferably C₁-C₄ alkyl), C₆-C₁₂ aryl, or arylalkyl (wherein the alkyl group contains 1-4 carbon atoms, and the aryl group contains 6-12 carbon atoms).

Reduction of the double bond can be carried out by catalytic reduction in the presence of a Pd—C, Raney-Ni, and/or Pt/C catalyst. The catalyst can be present to a concentration of about 5% to about 10% by weight Hydrogen for the catalytic reduction is present in the range of about 0.1 kg/m² to 20 kg/m² pressure; preferably 5-10 kg/m² pressure. The reaction is carried out in the presence of a solvent. The solvent used for the reaction can be selected from the group comprising halogenated hydrocarbons, a C₆ to C₁₄ aromatic hydrocarbon, a C₁ to C₅ alcohol, a C₂ to C₇ ester, and a C₄ to C₇ ether, a C₁ to C₅ carboxylic acid, cyclic ether, water and a suitable mixture of these solvents; preferably methanol, isopropyl alcohol, dichloromethane, toluene, ethyl acetate, diethyl ether. The reaction temperature is generally from about 15° C. to about 100° C.; preferably about 20° C. to about 40° C. The reaction time is generally about 1 hour to 5 hours; preferably about 1 hour to 3 hours. Typically, the amount of catalyst used is about 2 to about 30 gram per 100 gram of the compound of formula IV; preferably about 5 to about 20 gram per 100 gram of the compound of formula IV.

Compound of Formula VII as described above can be converted to ramelteon.

Other amines similar to compound VII can be prepared by using one of the amines or cyanates listed above (compound VII-a):

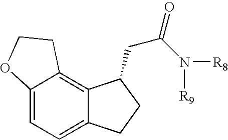

wherein each of R₈ and R₉ is independently selected from hydrogen, C₁-C₂₀ alkyl, C₆-C₁₂ aryl, alkylaryl (wherein the alkyl group contains 1-4 carbon atoms, and the aryl group contains 6-12 carbon atoms), optionally substituted with oxygen, nitrogen or halogen, wherein R₈ and R₉ are substituted in such a way so as to give a chiral compound.

Compound VII-a can be present in isolated or purified form, such as by HPLC, from other compounds (including Compound IV, the amine used in the reaction and/or ramelteon). Compound VII-a can be in a mixture (composition) comprising at least about 20% or at least about 40%, or at least about 60% or at least about 80% by weight of compound VII-a, and preferably at least about 90%, more preferably at least about 95% by weight of compound VII-a.

Compounds VII and VII-a can be converted to ramelteon by hydrolysis to obtain the corresponding free acid, and further converted to ramelteon using any process known in the art, for example, as described in PCT application no. PCT/US08/65590.

Ramelton can be prepared from intermediate II. A process for the preparation of ramelteon comprises the following steps:

(a) hydrolysis of compound of formula TI in presence of base or acid,

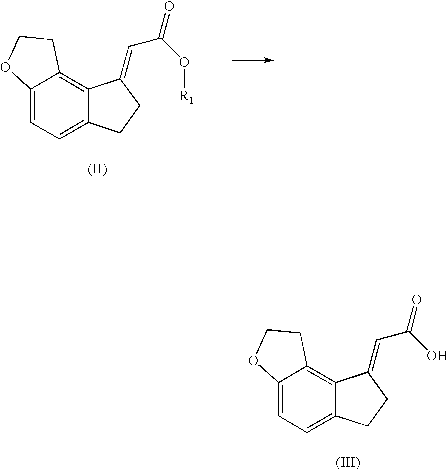

wherein R₁ is C₁ to C₄ straight or branched alkyl; preferably R₁ is methyl or ethyl.

The solvent for this step can be C₁-C₅ alcohol, preferably methanol and ethanol and more preferably methanol in presence or absence of water. The preferred base for hydrolysis is selected from alkali metal carbonates, hydroxides or hydrides, for example potassium bicarbonate, sodium bicarbonate, potassium carbonate, sodium carbonate, sodium hydroxide, potassium hydroxide, sodium hydride and potassium hydride; and is most preferably sodium hydroxide. Preferably, the acid is selected from sulfuric acid and hydrochloric acid. The reaction is carried out at temperatures from about 10° C. to about 40° C., preferably the temperature is about 25° C. The reaction time is generally from about 3 to about 8 hours, preferably about 5 to about 6 hours.

(b) condensation of the compound of formula III with a chiral compound to give a compound of formula IV:

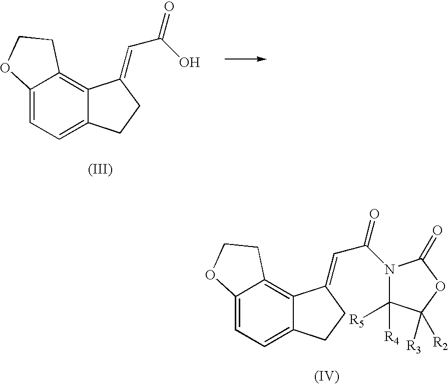

The organic base used for this condensation can be of the formula:

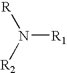

wherein R, R₁ and R₂ are each independently selected from C₁-C₈ alkyl, C₆-C₁₂ aryl and alkylaryl (wherein the alkyl group contains 1-4 carbon atoms, and the aryl group contains 6-12 carnob atoms. Examples of such amines can be triethylamine, trimethylamine, diisoproyl ethyl amine, tributyl amine, tripropylamine, more preferred is triethyl amine. An acid chloride can be added to the reaction mixture. The acid chloride can be pivalolyl chloride, methane sulfonyl chloride, p-toluene sulfonyl chloride, n-butyl chloride. More preferably pivaloyl chloride can be used. The acid chloride can be added at a lower temperature, ranging from about −5° C. to about 25° C. more preferably about 0-5° C. Then the chiral compound can be added. It can be a variously substituted 4-phenyl-2-oxazolidinone. A catalytic amount of 4-dimethyl amino pyridine, diethylamino pyridine, and/or dipropyl amino pyridine can be added. A catalytic amount is less than one mole and can be an amount of about 0.001 mol to about 0.2 mol. More preferably 4-dimethyl amino pyridine can be added. The reaction time can be generally about 20 to about 60 minutes; preferably 30-40 minutes. The temperature of reaction can be about 10° C. to about 35° C., more preferably about 20° C. to about 25° C.
(c) hydrogenation of a compound of formula IV in the presence of catalyst to give a compound of VII:

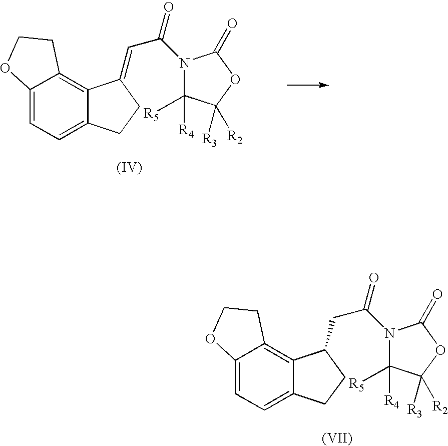

Reduction of the double bond can be carried out by catalytic reduction in presence of a catalyst selected from the group Pd—C, Raney-Ni, Pt/C, or combinations thereof. The catalyst is present in a concentration of about 5% to about 10% by weight. The hydrogen in the catalytic reduction can be in the range of about 0.1 kg/m² to 20 kg/m² pressure; preferably about 5-10 kg/m² pressure. The reaction is carried out in the presence of a solvent. The solvent for the reaction can be selected from the group of halogenated hydrocarbons, a C₆ to C₁₄ aromatic hydrocarbon, a C₁ to C₅ alcohol, a C₂ to C₇ ester, a C₂ to C₇ ether, a C₁ to C₅ carboxylic acid, cyclic ether (preferably C₄-C₁₂), water and a suitable mixture of these solvents; preferably methanol, isopropyl alcohol, dichloromethane, toluene, ethyl acetate, and/or diethyl ether. The reaction temperature can be generally about 15° C. to about 70° C.; preferably about 20° C. to about 40° C. The reaction time is generally about 1 hour to about 5 hours, preferably about 1 hour to about 3 hours. Typically, the amount of catalyst used can be about 2 to about 30 grams per 100 grams of the compound of formula IV, preferably about 5 to about 20 grams per 100 grams of the compound of formula IV;

The compound of formula VIII can be isolated as a free acid or as an amine additional salt, wherein when the amine salt is obtained acidification is required. The acidification is carried out, for example according to the procedure described in the PCT application no. PCT/US08/65590;

(d) hydrolysis of compound of formula VII in presence of base or acid.

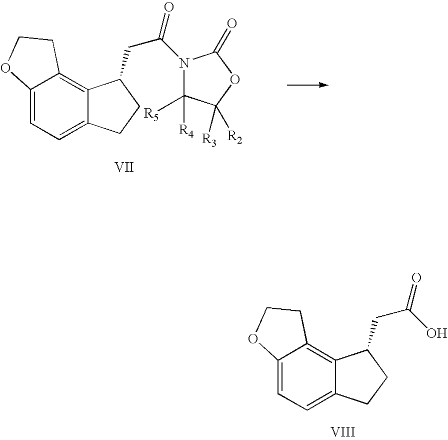

The solvent for hydrolysis can be alcohol, aliphatic or cyclic ethers, halogenated alkanes, aromatic hydrocarbons and mixtures thereof. Alcohols can be ethanol, methanol, and isopropyl alcohols, more preferably methanol. Ethers can be dioxane, tetrahydrofuran, tetrahydropyran, methyl tertiary butyl ether, diethyl ether. The more preferable ether is tetrahydrofuran. These solvents can be used alone or in combination with water in the ratio of 1:1. An oxidizing agent can be added, more preferably hydrogen peroxide can be added. The base for hydrolysis can be selected from alkali metal carbonates, hydroxides or hydrides, and mixtures thereof, for example potassium bicarbonate, sodium bicarbonate, potassium carbonate, sodium carbonate, sodium hydroxide, potassium hydroxide, sodium hydride and potassium hydride; most preferably lithium hydroxide. Preferably, the acid is selected from sulfuric acid and hydrochloric acid. The time of reaction can be about 2 to about 6 hours; preferable about 3 to about 4 hours.

(e) amination of the compound of formula VIII to obtain the compound of formula IX:

The acid chloride of formula VIII can be obtained by reaction of a compound of formula VIII with an acid chloride. The acid chloride can be selected from thionyl chloride, POCl₃, PCl₃, PCl₅, SO₂Cl₂, and oxalyl chloride. The compound of formula VIII can be isolated as the free acid or as an amine addition salt.

The reaction mixture can be distilled out and a suitable organic solvent can be added. The obtained reaction mixture can be treated with ammonia or an ammonia generating reagent such as urea to produce the compound of formula IX.

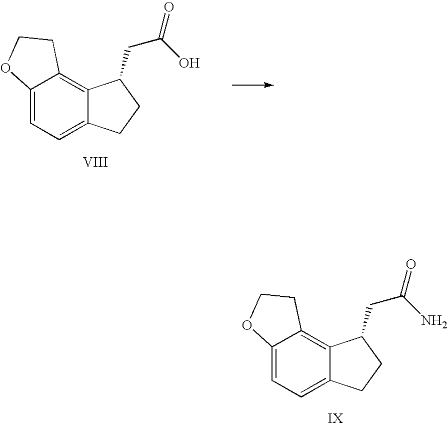

A suitable organic solvent can be selected from halogenated hydrocarbons, a C₆ to C₁₄ aromatic hydrocarbon, a C₂ to C₇ ester, and a cyclic ether (such as a C₅ to C₁₀), and a mixture of solvents thereof, preferably methanol, isopropyl alcohol, dichloromethane, toluene, ethyl acetate, and/or diethyl ether.

(f) reduction of the compound of formula IX with a ketone reducing agent to obtain the compound of formula X.

The compound of formula IX can be reduced in an organic solvent, followed by formation of a pharmaceutically acceptable salt of formula X.

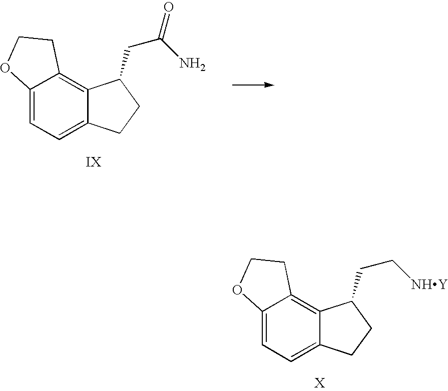

wherein Y is a pharmaceutically acceptable salt, which includes oxalates, sulphates, nitrates, phosphates, perchlorates, borates, hydrohalides, acetates (such as trifluoroacetate), tartrates, maleates, citrates, fumarates, succinates, palmoates, methanesulphonates, benzoates, salicylates, benzenesulfonates, ascorbates, glycerophosphates, and ketoglutarates.
(g) condensation of a compound of formula X with propionyl chloride and a base to produce ramelteon of formula I,

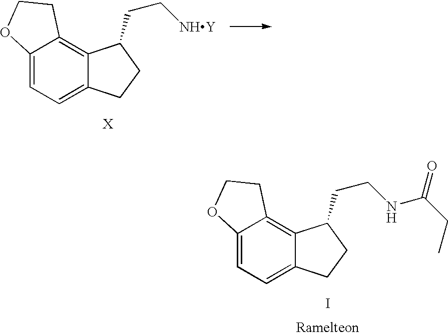

wherein Y is a salt selected preferably from oxalate, sulphates, nitrates, phosphates, perchlorates, borates, hydrohalides, acetates (such as trifluoroacetate), tartrates, maleates, citrates, fumarates, succinates, palmoates, methanesulphonates, benzoates, salicylates, benzenesulfonates, ascorbates, glycerophosphates, and ketoglutarates.

The organic base used for this condensation can be of the formula:

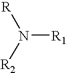

wherein R, R₁ and R₂ are each independently selected from C₁-C₈ alkyl, C₆-C₁₂ aryl and alkylaryl (wherein the alkyl group contains 1-4 carbon atoms, and the aryl group contains 6-12 carbon atoms. The organic base can be triethylamine. Other bases such as pyridine and piperidine can also be used.

A suitable organic solvent can be selected from halogenated hydrocarbons, a C₆ to C₁₄ aromatic hydrocarbon, a C₂ to C₇ ester, and a cyclic ether, and mixture thereof, preferably methanol, isopropyl alcohol, dichloromethane, THF, ethyl acetate, and diethyl ether.

This process is illustrated with 4-phenyl-2-oxazolidinone. Other chiral compounds, particularly the amines and cyanates listed above can also be used.

Having described the invention with reference to certain preferred embodiments, other embodiments will become apparent to one skilled in the art from consideration of the specification. The disclosures of the references referred to in this patent application are incorporated herein by reference. The invention is further defined by reference to the following examples describing in detail the process and compositions of the invention. It will be apparent to those skilled in the art that many modifications, both to materials and methods, may be practiced without departing from the scope of the invention.

### EXAMPLES

### Chiral HPLC Method Conditions

### Column: Chiral PAK ADH (250×4.6) mm, 5μ

Mobile Phase: n-Heptane:Ethanol (65:35)
Diluent: n-heptane:Ethanol (50:50)

### UV: 288 nm

Run time: 30 min
Inj. Vol: 10 μL
Flow: 0.8 ml/min
Column oven: 25° C.

### Sample Preparation: 1000 ppm

### HPLC Method Conditions for Chromatographic Purity:

Column: Xterra RP8, 3.5μ, 150×4.6 mm, Waters, P/N: 186000443 or equivalent.
Flow: 1.5 ml/min
Injection volume: 10 μl

### Detector: 217 nm

### Column Temperature: 30° C.

Equilibrium time: 10 minutes

### Diluent: Acetonitrile

### Example-1

### Synthesis of Intermdiate-II

A 60% suspension of sodium hydride in mineral oil (34.4 g, 1.43 mol) was added to dry toluene (3000 ml) under a nitrogen atmosphere, cooled, and stirred at 0-5° C. for 15-20 minutes. Triethyl phosphonoacetate (257.1 g, 1.148 mol) was added dropwise at 0-5° C. and stirred for 2 hr at room temperature. 1,2,6,7-tetrahydro-8H-indeno[5,4-b]furan-8-one (100.0 g, 0.5740 mol) was added, and the reaction mixture was heated to 90-100° C. and stirred under a nitrogen atmosphere for 15-18 hr. The reaction was monitored by HPLC. After completion of the reaction, the mixture was cooled and water was added slowly with stirring. The organic layer was separated, washed with brine, and concentrated under vacuum at 50-60° C. to leave crude product. Yield: 80-85%, Purity: 92-95% (By HPLC).

### Example-2

### Synthesis of Intermediate-III

A mixture of compound of formula II (ethyl (2)-1,2,6,7-tetrahydro-8H-indeno[5,4-b]furan-8-ylideneacetate) (100.0 g, 0.4098 mol) in methanol (2500.0) and water (500.0 ml) was hydrolyzed with sodium hydroxide (5.3 g, 1.4342 mol). The reaction mixture was stirred for 5-6 hrs at room temperature for hydrolysis. After hydrolysis the solvent was distilled off under vacuum. Water was added and extracted with dichloromethane. The aqueous layer was separated and acidified with hydrochloric acid drop wise under cooling. The precipitated solid product was filtered and dried under vacuum at 60° C. Yield 90-95%. Purity: 95-98% (Mixed isomer.)

### Example-3

### Synthesis of Intermediate-IV

The compound of formula III ((2)-1,2,6,7-tetrahydro-8H-indeno[5,4-b]furan-8-ylideneacetic acid) (10.0 g, 0.046 mol) was dehydrated with dichloromethane by distillation, the dehydrated material taken up in THF (200.0 ml) and cooled to 0-5° C. Triethylamine (10.3 g, 0.101 mol) and a catalytic amount of DMF (dimethylformamide) was added to the reaction mixture and stirred for 10-15 minute. Pivaloyl chloride (6.1 g, 0.0506 mol) was added slowly into the reaction mixture at 0-5° C. The reaction mixture was monitored for 3-4 hours, maintaining the temperature. After 3-4 hours, 4-phenyl 2-oxazolidinone (6.10 gm, 0.0506 mol) and a catalytic amount (0.8 g, 0.007 mol) of 4 (S)-dimethyl amino pyridine was added and stirred for 30-40 minute at 20-25° C. The reaction mixture was then refluxed for 20-24 hours at 45° C. Reaction progress was checked by TLC and HPLC. After completion of the reaction, the reaction mass was cooled to room temperature and the product isolated. Yield: 50-60%. Purity: 85-90%.

### Synthesis of Intermediate-IV:

A 60% suspension of sodium hydride in mineral oil (34.4 g, 1.4351 mol) was prepared in dry toluene (3000.0 ml) under a N₂ atmosphere at 0-5° C. and stirred for 15-20 minutes. A solution in dry toluene of (+)-(4S)-3-(bromoacetyl)-4-phenyl-2-oxazolidinone phosphonate (reference: J. Am. Chem. Soc. Vol. 120, No. 47, 1998, 12237-12254)(193.75 g 0.6888 mol) was added drop-wise at 0-5° C. and stirred for 2 hours at room temperature. The compound of formula VIII (100.0 g, 0.5740 mol) was added and the reaction mixture was heated to 90-100° C. under a N₂ atmosphere. The reaction was monitored by HPLC. After completion of reaction the reaction mass was cooled and water was slowly added. The organic layer was separated and washed with water and brine solution. The organic layer was distilled off under vacuum at 50-60° C. and the product isolated isolated in the form of crude oil.

### Example-4

### Synthesis of Intermediate-VII

A compound of formula IV ((4S)-4-phenyl-3-[(2)-2-(1,2,6,7-tetrahydro-8H-indeno[5,4-b]furan-8-ylidene)ethanoyl]-1,3-oxazolidin-2-one) (10.0 g) was hydrogenated in ethyl acetate (250.0 ml) in presence of 10% Pd/C (1.0) in an hydrogenator under 4-5 kg/m² pressure at 40° C. The reaction progress was monitored by HPLC. The reaction mixture was filtered on a celite bed after completion of the reaction and the solvent distilled off under vacuum at 50° C. and the product isolated. HPLC purity 97.2%; 97.1% (S); 2.9% (R).

### Example-5

### Synthesis of Intermediate-VIII

A compound of formula VII ((4S)-4-phenyl-3-[(2E)-2-(1,2,6,7-tetrahydro-8H-indeno[5,4-b]furan-8-ylidene)ethanoyl]-1,3-oxazolidin-2-one) (10.0 g, 0.027 mol) was dissolved in THF:water (1:1) (50:50 ml) and stirred at 0-5° C. 30% Hydrogen peroxide (14 ml) was added drop wise and lithium hydroxide (2.3 g, 0.054 mol) was added into the reaction mixture. The reaction mixture was stirred for 3-4 hours at room temperature. The reaction was monitored by TLC and HPLC. After completion, the reaction was extracted twice with ethyl acetate and 10% sodium bisulphite and 10% sodium hydroxide solution was added into the ethyl acetate layer and stirred for 10-15 minutes. The aqueous layer was separated and acidified with HCl. The product was filtered and dried under vacuum at 50° C. Yield 55-60%. Purity 90%.

¹H NMR (CDCl3, 400 MHz): δ 6.98 (d, 1H, J=8.0 Hz, aromatic), δ 6.65 (d, 1H, J=8 Hz, aromatic), δ 4.51 (m, 2H, methylene), δ 3.50 (m, 1H, methine), δ 3.10 (m, 2H, methylene), δ 2.9 (m, 1H, methylene), δ 2.78 (m, 2H, methylene), δ 2.36 (m, 2H, methylene), δ 1.87 (m, 1H, methylene)

### Example 6

### Synthesis of Intermediate IX

### Method A:

A solution of the compound of formula (S)-VIII ((S)—N-[2-(1,6,7,8-tetrahydro-2H-indeno[5,4-b]furan-8-yl)ethanoic acid or (S)—N-[2-(1,6,7,8-tetrahydro-2H-indeno[5,4-b]furan-8-yl)acetic acid) (100.0 g, 0.4587 mol) and thionyl chloride (300 ml) was stirred for 3 hours at room temperature, and the reaction progress was checked by TLC. Excess thionyl chloride was distilled off, dichloromethane was added, and again distillation was performed to remove traces of thionyl chloride. Ammonia gas was bubbled into a solution of the acid chloride in dichloromethane until a quenched sample tested alkaline. The precipitated product was filtered and washed with water and 5% bicarbonate solution. Yield: 85-90%. Purity: 95-98%.

### Method B:

A solution of the acid chloride of the compound of formula VIII in dichloromethane, prepared as above, was added drop-wise with stirring to a 30-35% ammonia solution (500 ml). The precipitated product was filtered and washed with water and 5% bicarbonate solution. Yield: 85-90%. Purity: 93-96.

### Method C:

To a solution of the compound of formula (S)-VIII (10.0 g, 0.045 mole) in dichloromethane (100 ml) and triethylamine (5.55 g, 0.055 mole), cooled to between −10° C. and 0° C., a solution of ethyl chloroformate (5.57 g, 0.0527 mole) in dichloromethane (10 ml) was added drop-wise with stirring. Stirring was continued for 1 to 2 hours at −10° C. to 0° C., until TLC indicated that the formation of mixed anhydride was complete. The reaction mixture was added drop-wise to a solution of ammonia in dichloromethane at −10° C. to 0° C., and the mixture was stirred for 30-45 minutes. Dichloromethane was removed under vacuum and the product was treated with 5% sodium bicarbonate solution (50 ml) with stirring. The compound of formula IX was collected by filtration and dried under reduced pressure at 50 to 60° C. Yield: 80-95%. Purity: 97-99%.

### Purification of Intermediate IX:

A solution of compound IX (100 g) in isopropanol (3000 ml), prepared by Method B above, was heated to produce a clear solution, and stirred for 30-35 minutes. The solution was decolorized with charcoal, filtered, and concentrated to 2000 ml, and then cooled to 15-20° C. The precipitated product was filtered and washed with cold isopropanol and dried. Yield: 80-85%. Purity: 98-99%. Recrystallization from ethanol and acetonitrile yielded comparable results.

### Example-7

### Synthesis of Intermediate-X

### Process-I (Hydrochloride Salt)

Sodium borohydride (74.2 g, 1.9631 mmol) was added into a stirred solution of BF3 etherate (247.8 ml) in THF (1800.0 ml) at −10° C. The reaction mixture was stirred for 2-3 hours at 0-5° C. and then the compound of formula IX (100 g, 0.4608 mol) was added and the reaction mixture was stirred at 40-45° C. for 6-7 hours. Reaction progress was monitored by TLC and HPLC. The reaction was quenched in 3600 ml water and 200 ml concentrated hydrochloric acid. THF was distilled out under vacuum at 40-50° C. The reaction mixture was diluted with toluene and basified with NaOH to pH 9-10. The organic layer was separated and washed with brine and sodium carbonate solution. The reaction mixture was concentrated and HCl gas was bubbled into it. The solid was precipitated, filtered and dried under vacuum at 50-55° C. Yield 70-80%. Purity 95-97%.

### Process-II (Oxalate Salt)

Sodium borohydride (74.2 g, 1.963 mmol) was added into a stirred solution of BF3 etherate (247.8 ml) in THF (1800.0 ml) at −1° C. The reaction mixture was stirred for 2-3 hours at 0-5° C. and then the compound of formula IX (100 g, 0.4608 mol) was added and the reaction stirred at 40-45° C. for 6-7 hours. Reaction progress was monitored by TLC and HPLC. The reaction was quenched in 3600 ml water and 200 ml concentrated hydrochloric acid. THF was distilled out under vacuum at 40-50° C. The reaction mixture was diluted with toluene and basified with NaOH to pH 9-10. The organic layer was separated and washed with brine and sodium carbonate solution. The reaction mixture was concentrated and an oxalic acid solution in methanol was added. The solid was precipitated and cooled to 0-5° C., filtered, washed and dried under vacuum at 50-55° C. Yield 80-85%. Purity 96-98%.

### Example-8

### Synthesis of Ramelteon

A mixture of the compound of formula X (100.0 g, 0.4175 mol) in THF (250 ml) and water (350.0 ml) was stirred at RT and then 30% sodium hydroxide solution (10.0 ml) was added. Propionyl chloride (68.74 g, 0.7432 mol) was added drop wise and stirred for 2-3 hrs. The reaction was monitored by TLC and HPLC. The solvent was distilled out and the aqueous layer extracted in ethyl acetate. The ethyl acetate layer was washed with brine and distilled out. The product was isolated and purified in ethanol. Yield: 50-60 gm. Purity 97-99%.

### Example-9

### Synthesis of Ramelteon

The compound of formula X (100.0 g, 0.3759 mol) was stirred in sodium carbonate (1120.0 g, 1.1277 mol) solution in water (600.0 ml) and dichloromethane (1000.0 ml) at room temperature. The reaction mixture was cooled to 10° C. to −5° C. Propionyl chloride (51.02 g, 0.5638 mol) in dichloromethane was added drop-wise into the reaction mixture and stirred for 1 hour. The reaction was monitored by HPLC and TLC. The organic layer was separated and washed with sodium bicarbonate and 10% brine solution. The organic layer was distilled out and the compound was isolated. The isolated compound was purified in alcohol. Yield: 92-96% & Purity: 99.5-99.9%.

What is claimed is:

1. A compound of the formula:

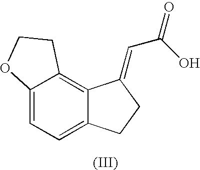

2. A mixture comprising the compound of claim 1, wherein the compound makes up at least about 20% by weight of the mixture.

3. A process for preparing the compound of claim 1, comprising:
hydrolyzing a compound of formula II in presence of base or acid to obtain the compound of formula III 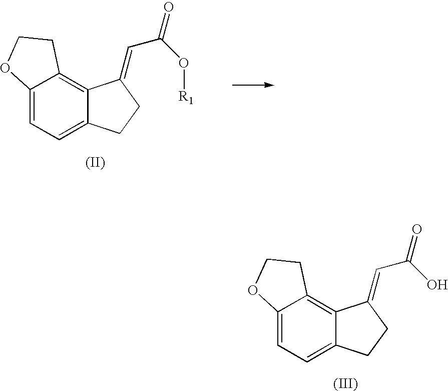
wherein R₁ is C₁ to C₄ (or C₁ to C₆) straight or branched alkyl.

4. The process of claim 3 wherein the straight or branched alkyl is methyl or ethyl.

5. A process for preparing ramelteon comprising:
a) preparing the compound of Formula III by the process of claim 3, and
b) converting compound III to ramelteon.

6. A compound of Formula IV: 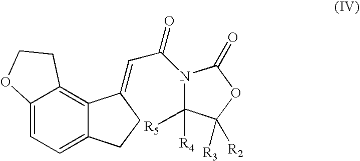
wherein R₂, R₃, R₄ and R₅ can be hydrogen, C₁-C₆ alkyl (preferably C₁-C₄ alkyl), C₆-C₁₂ aryl, or arylalkyl (wherein the alkyl group contains 1-4 carbon atoms, and the aryl group contains 6-12 carbon atoms), and R₆ and R₇ are selected from the group consisting of C₁ to C₄ alkyl.

7. A compound (Compound IV-a) the following structure: 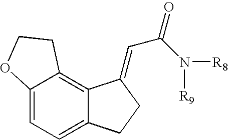
wherein R₈ and R₉ are substituted in such way to give an amine resulting from reaction with an amine or a cyanate selected from the group consisting of: (R)-1-Phenylethylamine, (S)-1-Phenylethylamine, (R)-1-(4-Methylphenyl)ethylamine, (S)-1-(4-Methylphenyl)ethylamine, (R)-1-(2-Methoxyphenyl)ethylamine, (S)-1-(2-Methoxyphenyl)ethylamine, (R)-1-(3-Methoxyphenyl)ethylamine, (S)-1-(3-Methoxyphenyl)ethylamine, (R)-1-(4-Methoxyphenyl)ethylamine, (S)-1-(4-Methoxyphenyl)ethylamine, (R)-1-(4-Chlorphenyl)ethylamine, (S)-1-(4-Chlorphenyl)ethylamine, (R)-1-(3-Chlorophenyl)ethylamine, (S)-1-(3-Chlorophenyl)ethylamine, (R)-1-(3-Bromophenyl)ethylamine, (S)-1-(3-Bromophenyl)ethylamine, (R)-1-(4-Bromophenyl)ethylamine, (S)-1-(4-Bromophenyl)ethylamine, (R)-1-(4-Fluorphenyl)ethylamine, (S)-1-(4-Fluorphenyl)ethylamine, (R)-1-(3,4-Dimethoxyphenyl)ethylamine, (S)-1-(3,4-Dimethoxyphenyl)ethylamine, (R)-1-(1-Naphthyl)ethylamine, (S)-1-(1-Naphthyl)ethylamine, (R)-1-Aminotetraline, (S)-1-Aminotetraline, (R)-1-Aminoindane,
(S)-1-Aminoindane, (R)-1-(2-Naphthyl)ethylamine, (S)-1-(2-Naphthyl)ethylamine, (R)-3-Methyl-2-butylamine, (S)-3-Methyl-2-butylamine, (R)-2-Hexylamine, (S)-2-Hexylamine, (R)-2-Heptylamine, (S)-2-Heptylamine, (R)-2-Octylamine, (S)-2-Octylamine, (R)-2-Nonylamine, (S)-2-Nonylamine, (R)-3,3-Dimethyl-2-aminobutane,
(S)-3,3-Dimethyl-2-aminobutane, (R)-1-Cyclopropylethylamine, (S)-1-Cyclopropylethylamine, (R)-1-Cyclohexylethylamine, (S)-1-Cyclohexylethylamine, (R)-1-Phenylpropylamine, (S)-1-Phenylpropylamine, (R)-1-Phenylbutylamine, (S)-1-Phenylbutylamine, (S)-1-Methoxy-2-aminopropane, (1R-trans)-2-(Phenylmethoxy)cyclopentaneamine, (1S-trans)-2-(Phenylmethoxy)cyclopentaneamine, (1R-trans)-2-(Phenylmethoxy)cyclohexaneamine, (1S-trans)-2-(Phenylmethoxy)cyclohexaneamine, (R)—N-Benzyl-1-phenylethylamine, (S)—N-Benzyl-1-phenylethylamine, (R)-1-Phenylethylisocyanate, (S)-1-Phenylethylisocyanate, (R,R)-Bis-(1-phenylethyl)amine, (S,S)-Bis-(1-phenylethyl)amine, and (R)-1-Phenylethylhydroxylamine, (S)-1-Phenylethylhydroxylamine.

8. A mixture comprising the compound of claim 6, wherein the compound makes up at least about 20% by weight of the mixture.

9. A process for preparing the compound of claim 6, comprising condensing the compound of formula III with a chiral compound to obtain a compound of formula IV: 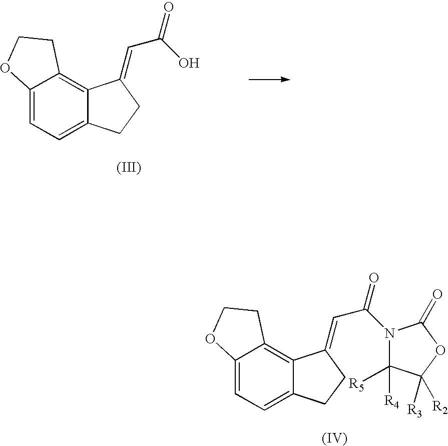
wherein R₂, R₃, R₄ and R₅ can be hydrogen, C₁-C₆ alkyl (preferably C₁-C₄ alkyl), C₆-C₁₂ aryl, or arylalkyl (wherein the alkyl group contains 1-4 carbon atoms, and the aryl group contains 6-12 carbon atoms), and in such way to give a chiral compound.

10. A process for preparing compound of claim 7 comprising reacting compound of formula III with an amine to get the compound of formula IV-a: 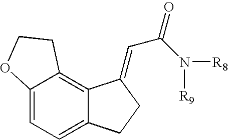
wherein each of R₈ or R₉ are hydrogen, C₁-C₂₀ alkyl, C₆-C₁₂ aryl, alkylaryl (wherein the alkyl group contains 1-4 carbon atoms, and the aryl group contains 6-12 carbon atoms), optionally substituted with oxygen, nitrogen or halogen, wherein R₈ and R₉ are substituted in such a way so as to give a chiral compound.

11. The process of claim 10, wherein R₈ and R₉ are substituted in such way to give an amine resulting from reaction with an amine or a cyanate selected from the group consisting of: (R)-1-Phenylethylamine, (S)-1-Phenylethylamine, (R)-1-(4-Methylphenyl)ethylamine, (S)-1-(4-Methylphenyl)ethylamine, (R)-1-(2-Methoxyphenyl)ethylamine, (S)-1-(2-Methoxyphenyl)ethylamine, (R)-1-(3-Methoxyphenyl)ethylamine, (S)-1-(3-Methoxyphenyl)ethylamine, (R)-1-(4-Methoxyphenyl)ethylamine, (S)-1-(4-Methoxyphenyl)ethylamine, (R)-1-(4-Chlorphenyl)ethylamine, (S)-1-(4-Chlorphenyl)ethylamine, (R)-1-(3-Chlorophenyl)ethylamine, (S)-1-(3-Chlorophenyl)ethylamine, (R)-1-(3-Bromophenyl)ethylamine, (S)-1-(3-Bromophenyl)ethylamine, (R)-1-(4-Bromophenyl)ethylamine, (S)-1-(4-Bromophenyl)ethylamine, (R)-1-(4-Fluorphenyl)ethylamine, (S)-1-(4-Fluorphenyl)ethylamine, (R)-1-(3,4-Dimethoxyphenyl)ethylamine, (S)-1-(3,4-Dimethoxyphenyl)ethylamine, (R)-1-(1-Naphthyl)ethylamine, (S)-1-(1-Naphthyl)ethylamine, (R)-1-Aminotetraline, (S)-1-Aminotetraline, (R)-1-Aminoindane, (S)-1-Aminoindane, (R)-1-(2-Naphthyl)ethylamine, (S)-1-(2-Naphthyl)ethylamine, R)-3-Methyl-2-butylamine, (S)-3-Methyl-2-butylamine, (R)-2-Hexylamine, (S)-2-Hexylamine, (R)-2-Heptylamine, (S)-2-Heptylamine, (R)-2-Octylamine, (S)-2-Octylamine, (R)-2-Nonylamine, (S)-2-Nonylamine, (R)-3,3-Dimethyl-2-aminobutane, (S)-3,3-Dimethyl-2-aminobutane, (R)-1-Cyclopropylethylamine, (S)-1-Cyclopropylethylamine, (R)-1-Cyclohexylethylamine, (S)-1-Cyclohexylethylamine, (R)-1-Phenylpropylamine, (S)-1-Phenylpropylamine, (R)-1-Phenylbutylamine, (S)-1-Phenylbutylamine, (S)-1-Methoxy-2-aminopropane, (1R-trans)-2-(Phenylmethoxy)cyclopentaneamine, (1S-trans)-2-(Phenylmethoxy)cyclopentaneamine, (1R-trans)-2-(Phenylmethoxy)cyclohexaneamine, (1S-trans)-2-(Phenylmethoxy)cyclohexaneamine, (R)—N-Benzyl-1-phenylethylamine, (S)—N-Benzyl-1-phenylethylamine, (R)-1-Phenylethylisocyanate, (S)-1-Phenylethylisocyanate, (R,R)-Bis-(1-phenylethyl)amine, (S,S)-Bis-(1-phenylethyl)amine, and (R)-1-Phenylethylhydroxylamine, (S)-1-Phenylethylhydroxylamine.

12. The process of claim 9, wherein R₂, R₃, R₄ and R₅ are substituted in such way to provide a 4-phenyl-2-oxazolidinone or a 5-phenyl-2-oxazolidinone.

13. The process of claim 9, wherein the process comprises combining compound of formula III with an organic base, adding an acid chloride to the mixture, a catalyst, and a chiral compound that gives a compound of formula IV.

14. The process of claim 13, wherein the organic amine is selected from the group consisting of trialkylamines: 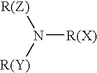
wherein R(X), R(Y) and R(Z) are each independently selected from C₁-C₈ alkyl group, more preferably C₁-C₄ alkyl group, C₆-C₁₂ aryl group, or alkylaryl (wherein the alkyl group contains 1-4 carbon atoms, and the aryl group contains 6-12 carbon atoms).

15. The process of claim 14 wherein the organic base is triethylamine.

16. The process of claims 13 wherein the acid chloride is selected from the group consisting of pivaloyl chloride, methane sulfonyl chloride, p-toluene sulfonyl chloride, and n-butyl chloride.

17. The process of claim 14 wherein the acid chloride is pivaloyl chloride.

18. The process of claims 13, wherein the acid chloride is added to the mixture at about −5° C. to about 25° C.

19. The process of claim 13, wherein the acid chloride is added at a temperature of about 0° C. to about 5° C.

20. The process of claim 9, wherein the chiral compound is selected from the group consisting of substituted 4-phenyl-2-oxazolidinones, 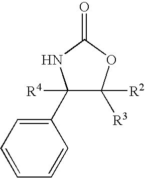
wherein R₂, R₃, and R₄ are each independently selected from the group consisting of hydrogen, C₁-C₄ alkyl, aryl and C₁-C₄ alkylaryl.

21. The process of claim 13 wherein the catalyst is selected from the group consisting of 4-dimethyl amino pyridine, diethylamino pyridine and dipropyl amino pyridine.

22. A process for preparing the compound of claim 6 comprising condensation of a compound of formula V 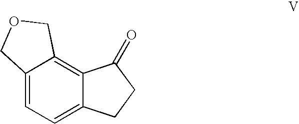
with a compound of formula VI 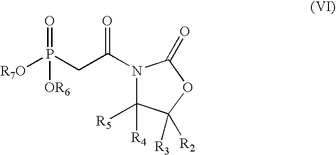
wherein R₂, R₃, R₄ and R₅ can be hydrogen, C₁-C₆ alkyl (preferably C₁-C₄ alkyl), C₆-C₁₂ aryl, or arylalkyl (wherein the alkyl group contains 1-4 carbon atoms, and the aryl group contains 6-12 carbon atoms), and R₆ and R₇ are selected from the group consisting of C₁ to C₄ alkyl group (or C₆-C₈ aryl group;)
in the presence of a base and a solvent.

23. A process for preparing the compound of claim 7 comprising condensation of a compound of formula V 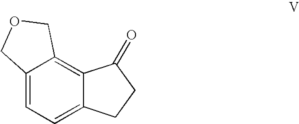
with a compound of formula VI 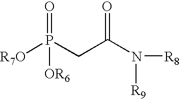
wherein each of R₈ or R₉ are hydrogen, C₁-C₂₀ alkyl, C₆-C₁₂ aryl, alkylaryl (wherein the alkyl group contains 1-4 carbon atoms, and the aryl group contains 6-12 carbon atoms), optionally substituted with oxygen, nitrogen or halogen, wherein R₈ and R₉ are substituted in such a way so as to give a chiral compound.

24. The process of claim 23, wherein R₈ and R₉ are substituted in such way to give an amine resulting from reaction with an amine or a cyanate selected from the group consisting of:
(R)-1-Phenylethylamine, (S)-1-Phenylethylamine, (R)-1-(4-Methylphenyl)ethylamine, (S)-1-(4-Methylphenyl)ethylamine, (R)-1-(2-Methoxyphenyl)ethylamine, (S)-1-(2-Methoxyphenyl)ethylamine, (R)-1-(3-Methoxyphenyl)ethylamine, (S)-1-(3-Methoxyphenyl)ethylamine, (R)-1-(4-Methoxyphenyl)ethylamine, (S)-1-(4-Methoxyphenyl)ethylamine, (R)-1-(4-Chlorphenyl)ethylamine, (S)-1-(4-Chlorphenyl)ethylamine, (R)-1-(3-Chlorophenyl)ethylamine, (S)-1-(3-Chlorophenyl)ethylamine, (R)-1-(3-Bromophenyl)ethylamine, (S)-1-(3-Bromophenyl)ethylamine, (R)-1-(4-Bromophenyl)ethylamine, (S)-1-(4-Bromophenyl)ethylamine, (R)-1-(4-Fluorphenyl)ethylamine, (S)-1-(4-Fluorphenyl)ethylamine, (R)-1-(3,4-Dimethoxyphenyl)ethylamine, (S)-1-(3,4-Dimethoxyphenyl)ethylamine, (R)-1-(1-Naphthyl)ethylamine, (S)-1-(1-Naphthyl)ethylamine, (R)-1-Aminotetraline, (S)-1-Aminotetraline, (R)-1-Aminoindane,
(S)-1-Aminoindane, (R)-1-(2-Naphthyl)ethylamine, (S)-1-(2-Naphthyl)ethylamine, R)-3-Methyl-2-butylamine, (S)-3-Methyl-2-butylamine, (R)-2-Hexylamine, (S)-2-Hexylamine, (R)-2-Heptylamine, (S)-2-Heptylamine, (R)-2-Octylamine, (S)-2-Octylamine, (R)-2-Nonylamine, (S)-2-Nonylamine, (R)-3,3-Dimethyl-2-aminobutane,
(S)-3,3-Dimethyl-2-aminobutane, R)-1-Cyclopropylethylamine, (S)-1-Cyclopropylethylamine, (R)-1-Cyclohexylethylamine, (S)-1-Cyclohexylethylamine, (R)-1-Phenylpropylamine, (S)-1-Phenylpropylamine, (R)-1-Phenylbutylamine, (S)-1-Phenylbutylamine, (S)-1-Methoxy-2-aminopropane, (1R-trans)-2-(Phenylmethoxy)cyclopentaneamine, (1S-trans)-2-(Phenylmethoxy)cyclopentaneamine, (1R-trans)-2-(Phenylmethoxy)cyclohexaneamine, (1S-trans)-2-(Phenylmethoxy)cyclohexaneamine, (R)—N-Benzyl-1-phenylethylamine, (S)—N-Benzyl-1-phenylethylamine, (R)-1-Phenylethylisocyanate, (S)-1-Phenylethylisocyanate, (R,R)-Bis-(1-phenylethyl)amin, (S,S)-Bis-(1-phenylethyl)amine, and (R)-1-Phenylethylhydroxylamine, (S)-1-Phenylethylhydroxylamine.

25. The process of claim 22 wherein the solvent is selected from the group consisting of C₆ to C₁₂ aromatic hydrocarbons, C₄ to C₈ aliphatic or cyclic ethers, C₂ to C₆ polar aprotic solvents, C₃ to C₆ ketones and combinations thereof.

26. The process of claim, 25 wherein the aromatic hydrocarbon is selected from the group consisting of toluene and xylene and combinations thereof.

27. The process of claim, 26 wherein the hydrocarbon is toluene.

28. The process of claim 25 wherein the cyclic ether is selected from the group consisting of dioxane, tetrahydrofuran and tetrahydropyran and combinations thereof.

29. The process of claim 28 wherein the cyclic ether is tetrahydrofuran.

30. The process of claim 25 wherein the polar aprotic solvent is selected from the group consisting of dimethylformamide and dimethylsulfoxide and combinations thereof.

31. The process of claim 22 wherein the base is selected from the group consisting of alkali metal hydrides and alkali metal hydroxides and combinations thereof.

32. The process of claim 31 wherein the alkali metal hydride is selected from the group consisting of sodium hydride and potassium hydride and combinations thereof.

33. The process of claim 31 wherein the alkali metal hydride is sodium hydride.

34. The process of claim 31 wherein the alkali metal hydroxide is selected from the group consisting of sodium hydroxide, potassium hydroxide and lithium hydroxide and combinations thereof.

35. A compound of formula VII: 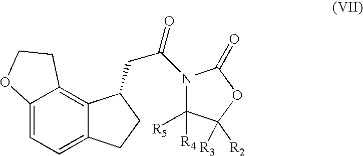
wherein R₂, R₃, R₄ and R₅ can be hydrogen, C₁-C₆ alkyl (preferably C₁-C₄ alkyl), C₆-C₁₂ aryl, or arylalkyl (wherein the alkyl group contains 1-4 carbon atoms, and the aryl group contains 6-12 carbon atoms).

36. The compound of claim 35, wherein R₂, R₃, R₄ and R₅ are substituted in such way to provide 4-phenyl-2-oxazolidinone

37. A mixture comprising the compound of claim 35, wherein the compound makes up at least about 20% by weight of the mixture.

38. A process for preparing the compound of claim 35, comprising reducing the alkene bond of a compound of formula IV: 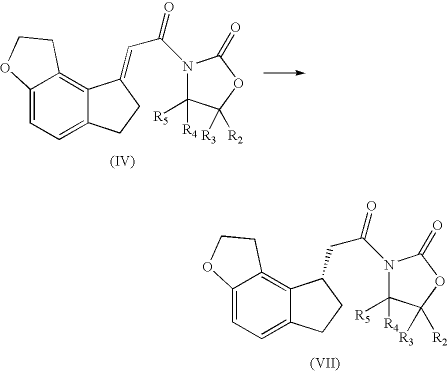
wherein R₂, R₃, R₄ and R₅ can be hydrogen, C₁-C₆ alkyl (preferably C₁-C₄ alkyl), C₆-C₁₂ aryl, or arylalkyl (wherein the alkyl group contains 1-4 carbon atoms, and the aryl group contains 6-12 carbon atoms).

39. A compound having the following formula: 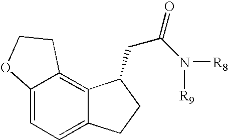
wherein R₈ and R₉ are substituted in such way to give an amine resulting from reaction with an amine or a cyanate selected from the group consisting of: (R)-1-Phenylethylamine, (S)-1-Phenylethylamine, (R)-1-(4-Methylphenyl)ethylamine, (S)-1-(4-Methylphenyl)ethylamine, (R)-1-(2-Methoxyphenyl)ethylamine, (S)-1-(2-Methoxyphenyl)ethylamine, (R)-1-(3-Methoxyphenyl)ethylamine, (S)-1-(3-Methoxyphenyl)ethylamine, (R)-1-(4-Methoxyphenyl)ethylamine, (S)-1-(4-Methoxyphenyl)ethylamine, (R)-1-(4-Chlorphenyl)ethylamine, (S)-1-(4-Chlorphenyl)ethylamine, (R)-1-(3-Chlorophenyl)ethylamine, (S)-1-(3-Chlorophenyl)ethylamine, (R)-1-(3-Bromophenyl)ethylamine, (S)-1-(3-Bromophenyl)ethylamine, (R)-1-(4-Bromophenyl)ethylamine, (S)-1-(4-Bromophenyl)ethylamine, (R)-1-(4-Fluorphenyl)ethylamine, (S)-1-(4-Fluorphenyl)ethylamine, (R)-1-(3,4-Dimethoxyphenyl)ethylamine, (S)-1-(3,4-Dimethoxyphenyl)ethylamine, (R)-1-(1-Naphthyl)ethylamine, (S)-1-(1-Naphthyl)ethylamine, (R)-1-Aminotetraline, (S)-1-Aminotetraline, (R)-1-Aminoindane, (S)-1-Aminoindane, (R)-1-(2-Naphthyl)ethylamine, (S)-1-(2-Naphthyl)ethylamine, R)-3-Methyl-2-butylamine, (S)-3-Methyl-2-butylamine, (R)-2-Hexylamine, (S)-2-Hexylamine, (R)-2-Heptylamine, (S)-2-Heptylamine, (R)-2-Octylamine, (S)-2-Octylamine, (R)-2-Nonylamine, (S)-2-Nonylamine, (R)-3,3-Dimethyl-2-aminobutane, (S)-3,3-Dimethyl-2-aminobutane, R)-1-Cyclopropylethylamine, (S)-1-Cyclopropylethylamine, (R)-1-Cyclohexylethylamine, (S)-1-Cyclohexylethylamine, (R)-1-Phenylpropylamine, (S)-1-Phenylpropylamine, (R)-1-Phenylbutylamine, (S)-1-Phenylbutylamine, (S)-1-Methoxy-2-aminopropane, (1R-trans)-2-(Phenylmethoxy)cyclopentaneamine, (1S-trans)-2-(Phenylmethoxy)cyclopentaneamine, (1R-trans)-2-(Phenylmethoxy)cyclohexaneamine, (1S-trans)-2-(Phenylmethoxy)cyclohexaneamine, (R)—N-Benzyl-1-phenylethylamine, (S)—N-Benzyl-1-phenylethylamine, (R)-1-Phenylethylisocyanate, (S)-1-Phenylethylisocyanate, (R,R)-Bis-(1-phenylethyl)amine, (S,S)-Bis-(1-phenylethyl)amine, and (R)-1-Phenylethylhydroxylamine, (S)-1-Phenylethylhydroxylamine.

40. A process for preparing the compound of formula: 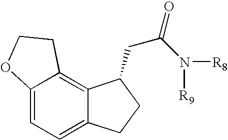
comprising reducing the alkene bond of a compound of formula IV: 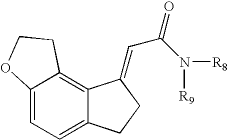
wherein each of R₈ or R₉ are hydrogen, C₁-C₂₀ alkyl, C₆-C₁₂ aryl, alkylaryl (wherein the alkyl group contains 1-4 carbon atoms, and the aryl group contains 6-12 carbon atoms), optionally substituted with oxygen, nitrogen or halogen, wherein R₈ and R₉ are substituted in such a way so as to give a chiral compound.

41. The process of claim 40, wherein each of R₈ or R₉ is substituted in such way to provide an amine selected from the group consisting of: (R)-1-Phenylethylamine, (S)-1-Phenylethylamine, (R)-1-(4-Methylphenyl)ethylamine, (S)-1-(4-Methylphenyl)ethylamine, (R)-1-(2-Methoxyphenyl)ethylamine, (S)-1-(2-Methoxyphenyl)ethylamine, (R)-1-(3-Methoxyphenyl)ethylamine, (S)-1-(3-Methoxyphenyl)ethylamine, (R)-1-(4-Methoxyphenyl)ethylamine, (S)-1-(4-Methoxyphenyl)ethylamine, (R)-1-(4-Chlorphenyl)ethylamine, (S)-1-(4-Chlorphenyl)ethylamine, (R)-1-(3-Chlorophenyl)ethylamine, (S)-1-(3-Chlorophenyl)ethylamine, (R)-1-(3-Bromophenyl)ethylamine, (S)-1-(3-Bromophenyl)ethylamine, (R)-1-(4-Bromophenyl)ethylamine, (S)-1-(4-Bromophenyl)ethylamine, (R)-1-(4-Fluorphenyl)ethylamine, (S)-1-(4-Fluorphenyl)ethylamine, (R)-1-(3,4-Dimethoxyphenyl)ethylamine, (S)-1-(3,4-Dimethoxyphenyl)ethylamine, (R)-1-(1-Naphthyl)ethylamine, (S)-1-(1-Naphthyl)ethylamine, (R)-1-Aminotetraline, (S)-1-Aminotetraline, (R)-1-Aminoindane, (S)-1-Aminoindane, (R)-1-(2-Naphthyl)ethylamine, (S)-1-(2-Naphthyl)ethylamine, R)-3-Methyl-2-butylamine, (S)-3-Methyl-2-butylamine, (R)-2-Hexylamine, (S)-2-Hexylamine, (R)-2-Heptylamine, (S)-2-Heptylamine, (R)-2-Octylamine, (S)-2-Octylamine, (R)-2-Nonylamine, (S)-2-Nonylamine, (R)-3,3-Dimethyl-2-aminobutane, (S)-3,3-Dimethyl-2-aminobutane, R)-1-Cyclopropylethylamine, (S)-1-Cyclopropylethylamine, (R)-1-Cyclohexylethylamine, (S)-1-Cyclohexylethylamine, (R)-1-Phenylpropylamine, (S)-1-Phenylpropylamine, (R)-1-Phenylbutylamine, (S)-1-Phenylbutylamine, (S)-1-Methoxy-2-aminopropane, (1R-trans)-2-(Phenylmethoxy)cyclopentaneamine, (1S-trans)-2-(Phenylmethoxy)cyclopentaneamine, (1R-trans)-2-(Phenylmethoxy)cyclohexaneamine, (1S-trans)-2-(Phenylmethoxy)cyclohexaneamine, (R)—N-Benzyl-1-phenylethylamine, (S)—N-Benzyl-1-phenylethylamine, (R)-1-Phenylethylisocyanate, (S)-1-Phenylethylisocyanate, (R,R)-Bis-(1-phenylethyl)amin, (S,S)-Bis-(1-phenylethyl)amine, and (R)-1-Phenylethylhydroxylamine, (S)-1-Phenylethylhydroxylamine.

42. The compound of claim 39, wherein R₂, R₃, R₄ and R₅ are substituted in such way to provide a 4-phenyl-2-oxazolidinone

43. A process for preparing the compound of claim 41 comprising the steps of:
a) mixing a compound of formula IV in a solvent,
b) adding a catalyst to the mixture,
c) introducing hydrogen into the reaction mixture,
d) reacting the mixture, and
e) isolating the product.

44. The process according to claim 43 wherein the solvent of step (a) is selected from the group consisting of C₅ to C₁₂ halogenated hydrocarbons, C₆ to C₁₄ aromatic hydrocarbons, C₁ to C₅ alcohols, C₂ to C₇ esters, and C₄ to C₇ ethers, C₁ to C₅ carboxylic acids, C₄ to C₈ cyclic ethers, water and a suitable mixture of these solvents.

45. The process according to claim 44 wherein the solvent is selected from the group consisting of methanol, isopropyl alcohol, dichloromethane, toluene, ethyl acetate and diethyl ether.

46. The process of claim 43 wherein the catalyst is selected from the group consisting of Pd—C, Raney-Ni, and Pt/C.

47. The process of claim 43 wherein the catalyst is present in about 5% to about 10% w/w.

48. A process for preparing ramelteon comprising hydrolyzing a compound of formula II 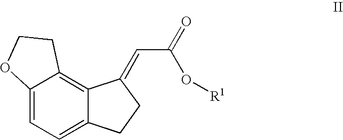
wherein R₁ is C₁ to C₄ (or C₁ to C₆) straight or branched alkyl, in the presence of base or acid to form ramelteon:

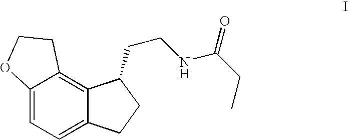

49. The process of claim 48 wherein R₁ is selected from the group consisting of methyl and ethyl.

50. A process for the preparation of ramelteon comprising the steps:
a) hydrolysis of a compound of formula II to produce the compound of formula III: 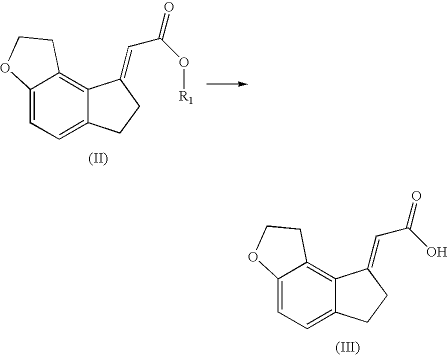
wherein R₁ is selected from the group consisting of C₁ to C₄ (or C₁ to C₆) straight or branched alkyl,
b) condensation of the compound of formula III with a chiral compound to yield a compound of formula IV: 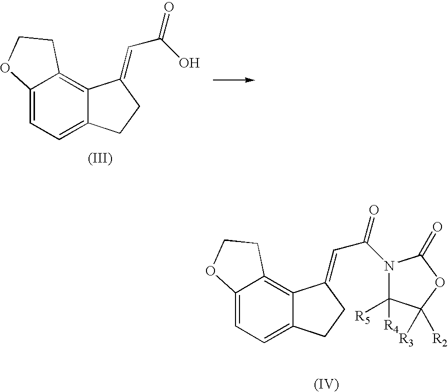
wherein R₂, R₃, R₄ and R₅ can be hydrogen, C₁-C₆ alkyl (preferably C₁-C₄ alkyl), C₆-C₁₂ aryl, or arylalkyl (wherein the alkyl group contains 1-4 carbon atoms, and the aryl group contains 6-12 carbon atoms),
c) hydrogenation of the alkene bond of the compound of formula IV to yield a compound of formula VII: 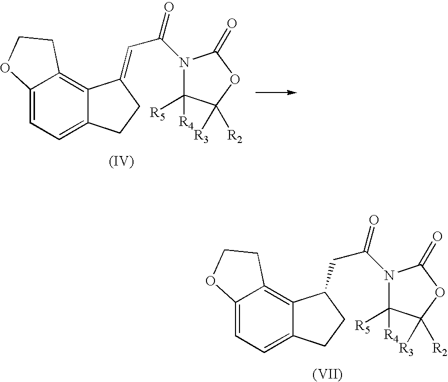
wherein R₂, R₃, R₄ and R₅ can be hydrogen, C₁-C₆ alkyl (preferably C₁-C₄ alkyl), C₆-C₁₂ aryl, or arylalkyl (wherein the alkyl group contains 1-4 carbon atoms, and the aryl group contains 6-12 carbon atoms),
d) hydrolysis of the compound of formula VII to produce the compound of formula VIII: 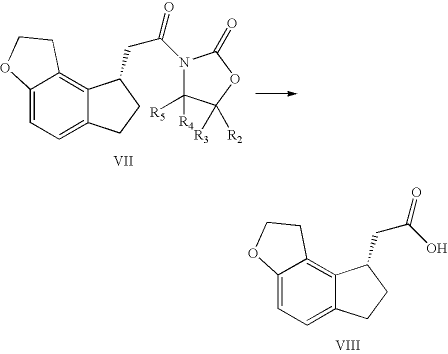
wherein R₂, R₃, R₄ and R₅ can be hydrogen, C₁-C₆ alkyl (preferably C₁-C₄ alkyl), C₆-C₁₂ aryl, or arylalkyl (wherein the alkyl group contains 1-4 carbon atoms, and the aryl group contains 6-12 carbon atoms), and in such way to provide a chiral compound;
e) amination of the carboxylic acid group of the compound of formula VIII to yield the amine compound of formula IX: 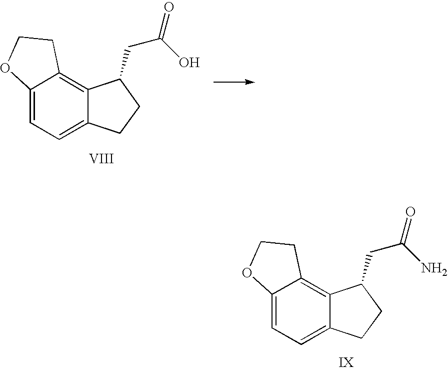
f) formation of the salt of the compound of formula IX to yield the compound of formula X; 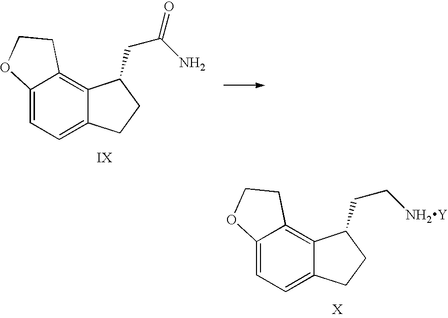
wherein Y is a pharmaceutically acceptable salt selected from the group consisting of oxalates, sulphates, nitrates, phosphates, perchlorates, borates, hydrohalides, acetates, tartrates, maleates, citrates, fumarates, succinates, palmoates, methanesulphonates, benzoates, salicylates, benzenesulfonates, ascorbates, glycerophosphates, and ketoglutarates.
and
g) condensation of the compound of formula X with propionyl chloride to yield the ramelteon of formula I 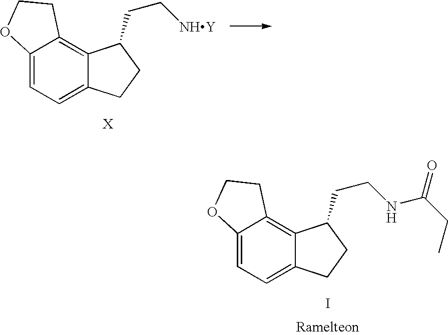
wherein Y is a pharmaceutically acceptable salt selected from the group consisting of oxalates, sulphates, nitrates, phosphates, perchlorates, borates, hydrohalides, acetates, tartrates, maleates, citrates, fumarates, succinates, palmoates, methanesulphonates, benzoates, salicylates, benzenesulfonates, ascorbates, glycerophosphates, and ketoglutarates.
